SEQUENCE LISTING (29 sequences)

SEQ ID NO: 1 - DNA, 1222 bp, Homo sapiens
aaaccccgtctctactaaaaatacaaaaaaatttgtcgggcatggtggcgggtgcctgtaatcccagctacttgggaggctgaggtaggagaacaccttgaacccaggaggtggaggttgcagtgagccgagattgcaccagtgcactccagcctgggtgacagcaagagactccaccacaaaaaaaaaaaaaaaaagcccagcttggtgcagtgactcacacctgtaatcactttgggaggctgagacaggaggattgcttgagcccaggagctcaagaccagcctgggcaacagagtaaggctttgtcactacaaaaagcaaatcaggtgtgtgtctgtagtctcggctactggagaggctaaggcaggagggctgtttgagcacgaggtcacggatgcataattgtgccactgcactccagcctgggtgaccaagtgagactctgtttcaaaaaaacaaaacaaaacaacaacaaaaaaacgggccccgatacctgaactagtgtctgttaccttgtggcaaaagagactttgcaagatgtgattaagtgtgtgcaccctgatataaagagattatcctggattatataaatgcgcccaatctaatcatgaggattttttatttttgagacgaagtctcactccgtcacccaggctggagtgcagtggtgtcatctcagctcactgcaacctctgcctcccaggttcaagctattctgccgtgtcagcctcccaagtagctagagtgcagtggcacaatcttggctcactgcaacctctgcctcctgggtttaagcaattctcctgcctcagcctcccgagtagctgggattataggtgcccaccaccacgcccggcttatttttgtttttttagtacagacggggtttcaccatgttggtcaggctggtctcaaactcctaacctcaggtgatctgcccgctttggtctcccaaagtgctgggattataggtgtgagccaccaaacctggctcacatgagtttttaaaagcagagacccggccaggcgtggtggctcacgcctgtaatcccagcactttgggaggccaatgtgggtggatcacgaggtcaggaaatcgagaccagcctggctaacacggtgaaaccccatctctactaaaaatacaaaaaattagccaggcgtggtggcgggcgcctgtagtcccagctactcgggaggctgaggcagaagaatggca

SEQ ID NO: 2 - DNA, 1534 bp, Homo sapiens
tgaaatgataattactaaatgctttcagaaaaatctagcggtgagggattggagaagcagaggggaaagtaagtcactttaaacatgtagcctaggctgacggggagagagtaggggcatggtgtgttccacaggtcatgctgggggctgacagtagaaagggaatggagttcctggagagggaatagttgtaaattattttcgtatacaataaagaaatagagttgattctgaatgccggaagagataaagggaacactgatagaatggaaatatacaaccaaatgcccacattaacaaagtggggaaagaaaatgtatatccaagagatcacactagcaaacactaggaagagggaaaaataataaacaacagaataaaataagtcataagcataaggttacattaagacattaaaatagaatacgtaagcttctccttcctcacactcagtgttttcttggctcagggaagtgactctctgttgtctgttgatcgtctctctcccatctgcggctttcaaactccttcaagaactcctattatttgcatgttagttgcctagatctgtcctccaggtctctctgcttccctcctgatttccatcactctgtgctgcatctccatagttgcacctgtttcttcttccatttggtgttgatcagctggtggtgaccagctcgcttgctccaggcctcaacagcaattttaacagactatttggagtcatctaccgaatttttgacttgaaaatattttttagttctagggtaccttttttgcacttggaaatcttcaagtgttcttatgtctttttggttcaagaggtagcatactgtagtggttcacagagtgggttctggagccagacttaaagtgcctgggttcaaatcctagctgggcctcttccccaccatgagatcattgacaagttacttaatctctctgtacttccattttcctatctgtaaaataggggagaataggagtggagtagttatatgaaaaactagaacttcttcataatctccattttctgtgtcctactctgtgaccacaattttcatcttcctagctgacttcttcaactatcacaactctgattatccttgggccctacttggctttctgtcattctacccaagcttcaagactactgtaagtaactatataataagtaactttacccactccttgcatgcatcatcaagtccctgccccatctcacttttttttttttttcagacggagtctctctgtagcccaggttggagtgcagtggcacgatctcggctaactgcaagctccacctcctgggttcatgccattctcctgcctcagcctcctgagtagctgggactacaggcgcccaccactacgcccggctaattttttgtatttttagtagagatggagtttcaccgtgttatccaggatagtcttgatctcctgaactcctgatccatccccctcagcctcccaaagtgctgggattacagacgtgagccacagcacccagcctcacttttttact

SEQ ID NO: 3 - DNA, 1026 bp, Homo sapiens
caagatttctaatgtatatggttttcacttaggaccttttttctgaatcatggtcaaaatatatagacgaataaaaatgtaaaactttttacacataatcccacttatttatgactaagcacttatattagtaaatttttttttatttgcaagtgacagcttatctcaaagtagcataagagagggagagagagaggaaagaaagaacctattggctcataaaatgcttgagtaagactcagaaacagatgataacagcttcaggcatggctagatccaagggccattagggtgttttatctcagtgtttcttggacaggcatattagccattggtagctcaaggcataccttatatttgcacagcagcccacatgaggaaagatcacctcccctgacatttctggctgaaagagcccagggaacagtctgacttgttcagcttgtgtcccgtgctcctccctgggcctgtcactgtgtccatgaggatgatgtcatctatctgatgagaccagcctggccaacatgatgaaaccccgcctctactaaaaatacaaaaattagctgggtgtggtggtgggcgcctgaagtcccagttacttggaaggcagaggcgtgagaattgcttgaacctgagaggtggaggttgcagttagccgagattgtgccactgccctccagcctgggcgacagagctagactctgtctcaaataaaacaaaacaaaataaaataaaataaaataataaataaataaataaataaataaactaaataatctgcttgatgaggcctgagagacacgtcctcacgtccctaaccattctacaactcaaatagtgtcagtctgcctttgcatcaatgtctcctcatttctacaaatagtaagaaaaatattcttctggtttatttcttaacgctgtcatgtaggtaaattgatgatcaggtgattgccttccgtcgtgtgcttgatttttctgggagtgcagtgttgcatggcaaagacaggtagagacatctacaatgtt

SEQ ID NO: 4 - DNA, 1285 bp, Homo sapiens
atctcaactgtattagtagaagagaaattgctgagttaaggggtattttgatgcagatttccaaattatcgtctaaaaaggttgcaccgatttatatacttattatctctccaaatatctgggtattattattataatttttttaaacctccaagcttcataagtaaaaatggcacatttattggttttaattagcatttttatttcttttgatgtataatattttaattagtttattgaaaatctctgtccttcctttttgaatttagtttatatcttttgcatattttaaagtttagattgttgtcttttctgttattaatttataaaatatttgtgttgagtgtgtgagtgcatgtgcatgcatgtgtatgcgtatgtgtgtgtttgtaacattttcatcagttttttgcttgccctctatttatggtattttcagatgtgattagatttatttggtctttgacattatgatttctgtctctatcattgagtttagaaggccaatattattcctacctcattaccaaagaaatacttaattctatctgcttacagatttaacttttttctttttccttttaaatctctaatacatttggaatttaattttcttactgattaaactttacttgtatatgtttatattttataatgataatattaaaccatttgtaatgcttaaattaaatattaaatggtcataacaccatttatttaaagcctattctttctttcaggtttgatatactgactttttcatcatgttgataatcttgtgatatattcctgttaatgctccattggtttctacattttttatcctgttcttataagttgtctgtctcctcctgtggctgttctattttattttaattactgaaactttattggacaatagtaagctcagaagagaatatctgtaggacaacagtaggctcaaggagaaatcatctgttctcctaatcttttactatttttcgtattttcctgactgatcttcagtatttctggaaaattgacaagcaatgcagattttggataatagtgcaggcttcactttcattgtgcctgggttcaaatcccagcttcttagctgactggcaatgtgacccagcaaatttatttactttggcatggctcagttttcttatctgtaaagtaagggtaatagtagtccctatattataagtttattatgatgattgtgtgggtggaacccgtgtgtggcacacaccggcacttagtgagaagttagctttgccattttcctttattcttcact

SEQ ID NO: 5 - DNA, 1408 bp, Homo sapiens
atgacccttggagaatgcattatcaaagcagagtcctgcagcctgaaagtaaacagcatctacttagaaatacagtgacatgtaaactttccccaaatcaacagtgaaaccagtagcaataactttgatgaaacaaaggggcattgccgagctgtcaggacacaacagagcacaacagaacagccccaggtaaatattgataaattaggttgatctgtgtattcttagtcatgggaagctggggcagataacgaaaccttgtatgtgtcctttatagctctgtctgatgagtttgggcgacggagtcttgctctgtcgcccaggctggagtgcaatggcgtgatctcagctcactgcaacctctgcctccctgattcaagtgattctcctgcctcagcctcctgagtagctgggattacaggcacgtgccgccacgccaggctaatttttgtatttttagtagagacagggtttcaccatgttgatcaggctggtctcgaactcctaaactcatgatccgcccacctcagcctcctgaagtgctgggattacaagcctgagccaccgtgcccagccttgtttttttttaagtgatttttgagggcagttttatgggcagaaaagactcttcgtgaatttattttgattttaaaattactattcaatttttcaaagttaaaaatgtaaaaaatctagtttattataaattgaagaccagcttaaaaatcatataataccatttatatgtctagcacattttaacaaacttcaacactaggttactccacttacaaatttggcaaactaaatattttaaggtattttaaaccgcatttatacatttactgtatttgatttttcttcttaagaactttggctaaaaccttcccaagtaatgaattaattcttataaatttaagttctcttagaaattttgacgctacatataagcttaaatattggatataaacttagtaaggatttcacttaaaatcgtaaatctaaacaatttccaattatacctttttaaaaaagagttacatgacttattctaatgggtcaatattttaggcatcacaaatacataattgcaaatatatagattccttaaattataccatggtcttgattcttattacatagatgtgtacatacctttctacccaggtctaaataccactgaattcaaaatgagtcttgcattctaagtacttcgaaattcttaatgggtattaaaaagtgacccagttgcttatggataaacctcataatttgccttgatacttggtcacaaattaaaggtcattctactgacaaattacttgtatacaatctacaaaacaaatagcatgcaattctcattacagcacacgtgttcagactttaacaaagctgtttgaagtctaca

SEQ ID NO: 6 - DNA, 1030 bp, Homo sapiens
agcaagggaaaataaagacttcttagtagtcataagttgagagacttcatccccagcagatacatttaaagctgcaaatgccaaaagaaggtttttacactgaaaggatatgatacaagattgaactagattggcaggaataaaagaacactagaagtggtcagtatctggataggtataaaagacttgtttatattatattttttctttaaaaggatatcatttttaaagcaaaaataataacaatatgttataggatttgagccatgtatgaaagtaaaatataagataatatgagcacgaatagcaggaggggcataaataaaattatactgttgtaattttctacatcactgtttgcaagtaacctgtgctaacttaaagataagtattataatctctagaacagccattaaaaagaacataaacaggtacaactaaaatgtcaatataggagataaaatcaggtacagctaaaatgtcaatataggagataaaatcaaatacaagacacacacacacacacacacacacacacacacacacacacacacacacaatttgcccaaaggaaggaagggaaaaaatagaggaaagcagaataaataaggcaaaaagaaaataaataccaatataggagacttaaaatcaactatatatcataatatacatatacatatacatatacatacacatatatttaatcctttttgtacaaaaggattaaaactcccaccatgactgagtggggtttgtcccagtaatgcaaagttggtttaatatttgacaatcagtcagtgtaagtgtattagttatctactgctgcacaacatattactccaaacctttcgtggcttacaacaatgaatatttatcatctcacacagtttctatgggtcaggatttaggagtagcttaactggatgattctggctcagcatctctcacgacattgcagtcaagataatgttagaggctgtagttatcttaatgcttgactgtgcttggaagatctgcttacaggatggct

SEQ ID NO: 7 - DNA, 1990 bp, Homo sapiens
cctaaaacttaaagtataataaaaaaataaataaataaataaaaaagaaaagaatgaactgaaagtgacaggttgccagagaggtggaaccacatggaatagagcagagaaagaaggagataaagatacagactacagcctattcagagtgacttacaaatgcaccgttctggataggatgccgtggggcagcctctcagcacacatatcagctaggctaattatatgttaattgtagttatttatcctggttttataatataatttctctggaagttgttagaatttttattctaactttaatgttctgaaatttcatatacatatggaataaattgctttggatttttatttgttttattttccatttgatttaatttggttttgtaaatcctgctcaacactgagtagggtcttttttctaatctcctaactttatttctctataaaggaatattctctgttatctcttaaaatattggcagctccgtattacgactaatcttttgaaactctatttagatgatttttgggaacttctgggtctgttctccaattctcttcccttttattatttctctgtctttagtttcagctttataattaattctactgccacatacattttgcactttggcctataaatttcacaatttgtgtttttttatttttatagttctagttggttcttctttatatccacctagtcatgttatggaataaaataccatggctccaaagacaaaaaagactgtctcttttattctttgcgtaacttaccagctccaactgtggcctgaacttctgctatttatcattgagaagtgagaagatatagaattagccccactgactgaacactagtttcctgtaatgctcttcattgctctccggcattcattaccagataaccaaggcatgtcccggagtatctaatgtaaactcacatattcactattcatcctggggcgaggtccatagatttctgctgcctgtccaaagaagcataggttgaactatcaaattgcttcaatgatccagggaattaattgctcttaaacctcctcctacttttctatactgcagaaatatcacatctaaagtaacaatatttcttgatcttttgtgctgcaattatcctcttcagcttctacttcctaatgactgctcagtttctatttaccaataattgctcataattgccataattctcttgcttttttaattttttaattttttatttttttgagacaaagtcttactctgtcgcccaagctgtagtgtagtggcgcgatctcagttcactgcaacttccacctcccgggttcaagcgattctactgcctcaacctcctgagtagctgggactacaggcatgcaccaccatgcctggctaatttttttttaatgtatttttagtagagacggggtttcactatgttggccaggctggtcttgaacagctgatctcgtgatccgccccctcagcctcccaaagtgctgggattacaggcgtgagccaccatgcccagccgccttttttttgtttgttttttgttttaaagagtgtgacttttagggatcgagtacgatggagctgccaggtgtggttattttgccaccttgaaacgagaatgtatcatcttatctctattgattgccagagttcaagaactagagcaacaggggtctcagcttttagcagcgatggcttttctagggaatttatttaaagcacttaaaatgatgcagtgtagtatgcacaaaaatttcaaataattataacttttgactcagcaaatccacttctgggaatttgaccaatattgttaatgataccattcagttaagatgtttagttaaggacatttattgcaatattatttaaaatagcaaagtattagaagccatctggatgttctttaaaatggttaaatgcattttggtctatttttactataaaatattatgcaactattataaagaatgaa

SEQ ID NO: 8 - DNA, 1078 bp, Homo sapiens
tgtttaacttaactttatatttactagtgggaatatttagttaccaacggtaacaggtaatgtaaaatgattatttggatgttcttggtaggaaaataataatcacattataaaatcagggctgaatttaattttgaagtcttaaaatggaagttaaatggtagcacaaagagcattcaacattgagctaagagcttacaaattcacagtctgattcattaattcacagcctggcattcatcatatgcctcagtttccactctgtaaaatgacaagattgagagtaaatgatcccaaggtttccctctagctctaaaaattctctattctgcacacatatggcactatattttaaagcacatacgcctcctgtgaagtgaaataaagtgaataagcacacggaaatggtacggtgattaaatttactcagagctaaaaatcattgcatattagccttacatatgaaaaaaaaacttgactaatgaatcaaacttgaaggatctgtcagtgtgcagttgtttgacacagaacaaggactgtgaaattgctgattttatcccagtgattcttgtatgggtcacacacagcaaaaagaggggtcagtaagtttcacattcatatatataagcttgtaaaaggaaagaaaaaggagtttcttttgacattgcaatgaactatatgagtcttaatgaacattactgttaaggcacccaagcaaattagcgtttccccacatgccttagcttgcaagaaattaaatttggcctaaataaattaaagcagtggacctgacacatctgatcatttctacctctcaattatttttcctttggtcaggttttcttggaagaaattattcattttgaaatgttgatattctggttggatctatctctgtgctgtatttttgcagctggagaagctatggactgtggatttcaagtgtgacactcactaagaaacccaaggagtcaagaacactattgcattcgtggggataatttcagaatcaggtttatccggaacctggatattcataccactactaagaagtgttgcaattcttttaggtatgatg

SEQ ID NO: 9 - DNA, 1408 bp, Homo sapiens
tttgcctttgtgatttataagtaatctgtgcacagatatttcagacagtatacatacattcttttttatttgaattcattaaaattcactcactggtttgggcatctattggtaagtgttttctgcaataattgttactatgatggctgccaaatagtgattcacaattttatcattgcttctacatttactggttgtcactctactgtaggaaataggtcctccacacctccatttactttgtttacttatttatgtatttattgatcagcagtttcacagatcattttaatttaaagatgatcatccattattcttattttgatgttcacattgtcctagcttaggccagtgggagccctttcaggctacctctttgctctttcactacttgcattttaaaagttttatgcataccttcctgccaaagttgtgacatttttaatatgaagaagtttttaattttgagaaaatctaattcattttttttgtggttttaatttctgtgtcctgagacacatttgcctacacacaagtcacaaagattttctcctatattttttatctaaaagctttatagttttgtttttatatttcagtctataatacatctcaaattaatttttgtgttttgtgtgaagtagggtctaggtatattgctttcccaaaggacagctagttttcctaacacgatttgttaagattattcattttcctaatgaatttttgaccaaacaaatcaaatggacatctaagtgtgtctctagtttttagccctttattctattactttaatctatttattggttctgtgccagtaaaataataatttgattacagcatattatagttaatcttgaggttatgtagtgtatgtctcccaagtttgttcatctttctcaagtttgctttggatagtctaggtccttaatatttctgtatgaattttaaaagcagggttgttggattttgatagaaattacatggaacctatagatcaatttgaggtaaaattgacatcttaacatttagtcttctatcaacatggcatatttctctatttcttcaagtcttcttaattcctttcagtaggtaactaaactggacactatacatctttggttaaacatgttcccctataggtggtatcaaaaatgtcacttcatatctttcacccttagctgagctgctagcagtgtactctatacactcacggttcatggtgtagccttgagatatggggagagttaatacaccgatttaggggctctccctttccctctcctttccaggactacccttagtcttctgagaggaagtgtttgcttcaagttctaacctccagttcttgccaaaaaaggctgcaggttttctgtgggagttttaattggcagtgtgacactgacta

SEQ ID NO: 10 - DNA, 1095 bp, Homo sapiens
aataagactctgcagagcctcactgcacttccttcctggaagttttacaaaggggttaatgagctctgcttaccctataaaatcaaatccacgggactgcagagcaagtgagtcataatatttgtggaataaatgaatgtagcatgacagtccctggatatgacttgccaacaattacagagggaagaaggtgaccctttaaatgccagttggatgacaagctctgtagaatttccttagtataggctcaatagaattctgagccttggcagaggatggtcagtattcaacatatggattttagtatcagatctccacacacaaggtctgtatgatctttgttataagaagaaggactatatgatctttgtatagccaaatacaagcgctttatgatctttgttataagaacaagctcttgtttgcatttgaatgattatttgtagtttcatagcactttaaaatgtcatattctacctgataaagctgtgggctatgtgttttgattttcgttttgtagctgaagaaactaagactcagaaagtttaaatagacagtgagtagtaggtgaggcaatggagcctaaactcccttcctgtgtgtcagtaaacatggccttgtgaattcaccgggagcctcagggctcatgaatgggctgaggtgccataaccattgtcctgggcgcaagctgcttgacaggttgctatacagtggatgttttctctcttctttttaaagtttgaaagcaatactttaaaagataatcaaaatgagcccttataattccatagcacctcccttctccattgctagtcactgggtgctagaagccaggggagaataaaaaggaaatattaaacagtgaagtagcaatcaaagcttcagctgggttcctttagaggagagagcttcagaaggggccaaagtctgggccccagaacttcctggtggcagagaaatgggcaggcggcctttcagctgatatctcagaccacagactcaagggacaattcaggcagctcccccagtaagccagccacaggcaaagcagaggccaataaccctgcacctggcttcctggtccttctctcccga

SEQ ID NO: 11 - DNA, 2095 bp, Homo sapiens
tcttcaaatttactatataatgcaaaattgctttccaaagtgtttaaaccagttacactcccaccagctgcgtaagaattgaagttaatctgtctctgctgaaacactttttatttttttaaaaaaaggaaggatgtgcaatgttatctcagtgatgttatattttcctctttccatatgcgtttttcatatgtgtttattagctatttaatttcctgttcatatcatttgctcatcttcctgttggattgtctctattcttactgatttgctcataagtcattttatattctaattactaaacctttgttgtttatagtggttgaaattatcttctcgaatactgtggatcaggggttggcaaactatggtccttgtttatggcctatttttgcacatcccaaactaattttaatgtaatctaatgtctttacatttttaaagattgcaaagaaagaagaaagcagcagcagcagcagtggcaacagagatgcatgtggcctgcaaagccgaaaatatttatcatctggctattcacaaaaaattgctaaccaccccccgcaacccgctatgactgttttctaacttagtttctatttttgttgtcattgaaatgtttcatttcaatgtagtataatgtatcattttaaagattataatattatagattatatgactctgcttctagctctctgatttcattgttctattcatatatcaaaatgccaataccacttttaaatttactagtgttccaaaataagtctgatatctggtagggcaaatctgtccccttccaacccattttctctcttaaacagttctgatccaacaacctccccacttggtcacaatgaccaaagttgattcaatgcatgcattaattttatatttatacctttcaactgccagtatataaaattcaaagttgtttccgcccctaccccccgccagctgattattgttattgctattcctgaaagggggtatttttcaatccatggcacttgtgtgcattagcaataattagataactttgctcaaaaaatccaactcattttgaagacatctgtttgactttctttgcccttctgctggcttctggtgcaggagctgagctcttaattaattattgcttatatcgggtagtaataccccctgaaaagtgaagcaagcttgcgctaaaggaagcatagaaaacagtacatttctgtaaacagacgacaatgtttactgaatgcttatttctttaaatgtcattgggtatataaaaccaagcttcttggagttctatccactaaactcaccgaatcattccaaattctaacttaatttcagcagtagagatgattcatagcaaagcagaaccaaatgggctgcagttggatttttaggtttctgtagttcgtgttttcaagtttttaaacagctttattaataaatttctttatttgtttgtctgtttatcgggcagactcccaagccagaataggctcagggagactccttaaacagctttactaaggcataatctaccgacttttaaattcgctcattgtaagtgttcaattcaatgatttttcacaaatttacagagttgtgcagctatcaccatgacccagttctagatcattccctttaacccaaaaagttccttcagactggtttgtagtcaattctccccaccccttggcaattgctaatctgctctctgtctctgtcaatttaccctttctggatatttaatataaatggaatcatacaatatgtagcctttgtgtctggcttctttcactctgttttagcagactatttttgaggttcatctatgtatcagtagttttttttcttcctttttattgctgaaaagtgttgaagtcaaataaaatatagagagatgaatttcttttctttttttcttttcttttttttttgagacagagtctcactgtgtcgcctaggctggtgtgcagtggagcaatctcggctcactgcaagctccgccttccaggtttacaccattctcctgcctcagcctcccgagtagctgggactacaggcgtccgccaccacgcttggctaattttttgtattt

SEQ ID NO: 12 - DNA, 1245 bp, Homo sapiens
tttactcaggagcttttgaacacagtagcattctagcagaaatgagccttgacaagttggttttttttttttttttttgaaatgtagtttcactcttgtcgcccaggctggagtgcaatggcgtgatgatctcagctcactgcaacatctgcctcccaggttcaagtgattctcccacgtcagcctcctgagtagctgggattacaggcacctgccaccatgcccagctaatttttgtatttttagtagcgatggggttttgacatgttgaccaggctggtctcaaactcctgacctcaggtgaaccgcccaccttggcctgaccaaagtgctgggattacaggcgtgagccatcatgccccgttgacatgtcggattctttaaagagaaattgtctctagacagcagaatatggactgcccactcagcccagatatctctcaataataataataataatagaagaagaaggagaaggaggaggaggagaaggagcagaaggagaaggagaaggagaagaaaggagaaggagaaggagaagaagaagaagaagaggaggaggaggaggaggaggaggaggacgaagaagaaaaggttctttaaagtgagaccttaaggaagttatgactgactgacccttactatgtagacccgcttttttttttggagacagtgtcttgctctgttgtccaggctggagtgcagtggcgtgatcatagctcactgcagcaatgaacttctgggcttaagtgatcctcctgccttaacctcctgaatagctaggactacgggtgcacaccaccatgcccagctaatttttttagttttatttttgtagagatgggggtctcactatgttcccccaggatggtcttgaactcctagcctcaaacaatcctttgatcctccctcctcagcctcccaaagggctgggattacaggcatgagctgctgctcccttagagacctgctttttcacataaattttaagttcctaaaacagactgtccggctcctcaggtagctcttaaccttgagttcctccagtcaccttgggctagctctggtccagccccagccctcttcagcagtgcccttgaccttgtctctcacccttgtctcctgcgttctagaggtcctgggctattcactcctggcttcaacccttccttacccttgactctggcttactcacaaccagtgctatccataaggagtgaatccctattgctttatttctacaaa

SEQ ID NO: 13 - DNA, 3923 bp, Homo sapiens
gaattcatctcaactgtattagtagaagagaaattgctgagttaaggggtattttgatgcagatttccaaattatcgtctaaaaaggttgcaccgatttatatacttattatctctccaaatatctgggtattattattataatttttttaaacctccaagcttcataagtaaaaatggcacatttattggttttaattagcatttttatttcttttgatgtataatattttaattagtttattgaaaatctctgtccttcctttttgaatttagtttatatcttttgcatattttaaagtttagattgttgtcttttctgttattaatttataaaatatttgtgttgagtgtgtgagtgcatgtgcatgcatgtgtatgcgtatgtgtgtgtttgtaacattttcatcagttttttgcttgccctctatttatggtattttcagatgtgattagatttatttggtctttgacattatgatttctgtctctatcattgagtttagaaggccaatattattcctacctcattaccaaagaaatacttaattctatctgcttacagatttaacttttttctttttccttttaaatctctaatacatttggaatttaattttcttactgattaaactttacttgtatatgtttatattttataatgataatattaaaccatttgtaatgcttaaattaaatattaaatggtcataacaccatttatttaaagcctattctttctttcaggtttgatatactgactttttcatcatgttgataatcttgtgatatattcctgttaatgctccattggtttctacattttttatcctgttcttataagttgtctgtctcctcctgtggctgttctattttattttaattactgaaactttattggacaatagtaagctcagaagagaatatctgtaggacaacagtaggctcaaggagaaatcatctgttctcctaatcttttactatttttcgtattttcctgactgatcttcagtatttctggaaaattgacaagcaatgcagattttggataatagtgcaggcttcactttcattgtgcctgggttcaaatcccagcttcttagctgactggcaatgtgacccagcaaatttatttactttggcatggctcagttttcttatctgtaaagtaagggtaatagtagtccctatattataagtttattatgatgattgtgtgggtggaacccgtgtgtggcacacaccggcacttagtgagaagttagctttgccattttcctttattcttcactctcgagcctaaaacttaaagtataataaaaaaataaataaataaataaaaaagaaaagaatgaactgaaagtgacaggttgccagagaggtggaaccacatggaatagagcagagaaagaaggagataaagatacagactacagcctattcagagtgacttacaaatgcaccgttctggataggatgccgtggggcagcctctcagcacacatatcagctaggctaattatatgttaattgtagttatttatcctggttttataatataatttctctggaagttgttagaatttttattctaactttaatgttctgaaatttcatatacatatggaataaattgctttggatttttatttgttttattttccatttgatttaatttggttttgtaaatcctgctcaacactgagtagggtcttttttctaatctcctaactttatttctctataaaggaatattctctgttatctcttaaaatattggcagctccgtattacgactaatcttttgaaactctatttagatgatttttgggaacttctgggtctgttctccaattctcttcccttttattatttctctgtctttagtttcagctttataattaattctactgccacatacattttgcactttggcctataaatttcacaatttgtgtttttttatttttatagttctagttggttcttctttatatccacctagtcatgttatggaataaaataccatggctccaaagacaaaaaagactgtctcttttattctttgcgtaacttaccagctccaactgtggcctgaacttctgctatttatcattgagaagtgagaagatatagaattagccccactgactgaacactagtttcctgtaatgctcttcattgctctccggcattcattaccagataaccaaggcatgtcccggagtatctaatgtaaactcacatattcactattcatcctggggcgaggtccatagatttctgctgcctgtccaaagaagcataggttgaactatcaaattgcttcaatgatccagggaattaattgctcttaaacctcctcctacttttctatactgcagaaatatcacatctaaagtaacaatatttcttgatcttttgtgctgcaattatcctcttcagcttctacttcctaatgactgctcagtttctatttaccaataattgctcataattgccataattctcttgcttttttaattttttaattttttatttttttgagacaaagtcttactctgtcgcccaagctgtagtgtagtggcgcgatctcagttcactgcaacttccacctcccgggttcaagcgattctactgcctcaacctcctgagtagctgggactaggtacctttactcaggagcttttgaacacagtagcattctagcagaaatgagccttgacaagttggttttttttttttttttttgaaatgtagtttcactcttgtcgcccaggctggagtgcaatggcgtgatgatctcagctcactgcaacatctgcctcccaggttcaagtgattctcccacgtcagcctcctgagtagctgggattacaggcacctgccaccatgcccagctaatttttgtatttttagtagcgatggggttttgacatgttgaccaggctggtctcaaactcctgacctcaggtgaaccgcccaccttggcctgaccaaagtgctgggattacaggcgtgagccatcatgccccgttgacatgtcggattctttaaagagaaattgtctctagacagcagaatatggactgcccactcagcccagatatctctcaataataataataataatagaagaagaaggagaaggaggaggaggagaaggagcagaaggagaaggagaaggagaagaaaggagaaggagaaggagaagaagaagaagaagaggaggaggaggaggaggaggaggaggacgaagaagaaaaggttctttaaagtgagaccttaaggaagttatgactgactgacccttactatgtagacccgcttttttttttggagacagtgtcttgctctgttgtccaggctggagtgcagtggcgtgatcatagctcactgcagcaatgaacttctgggcttaagtgatcctcctgccttaacctcctgaatagctaggactacgggtgcacaccaccatgcccagctaatttttttagttttatttttgtagagatgggggtctcactatgttcccccaggatggtcttgaactcctagcctcaaacaatcctttgatcctccctcctcagcctcccaaagggctgggattacaggcatgagctgctgctcccttagagacctgctttttcacataaattttaagttcctaaaacagactgtccggctcctcaggtagctcttaaccttgagttcctccagtcaccttgggctagctctggtccagccccagccctcttcagcagtgcccttgaccttgtctctcacccttgtctcctgcgttctagaggtcctgggctattcactcctggcttcaacccttccttacccttgactctggcttactcacaaccagtgctatccataaggagtgaatccctattgctttatttctacaaaggatcc

SEQ ID NO: 14 - DNA, 3718 bp, Homo sapiens
gaattctcttttgatgtataatattttaattagtttattgaaaatctctgtccttcctttttgaatttagtttatatcttttgcatattttaaagtttagattgttgtcttttctgttattaatttataaaatatttgtgttgagtgtgtgagtgcatgtgcatgcatgtgtatgcgtatgtgtgtgtttgtaacattttcatcagttttttgcttgccctctatttatggtattttcagatgtgattagatttatttggtctttgacattatgatttctgtctctatcattgagtttagaaggccaatattattcctacctcattaccaaagaaatacttaattctatctgcttacagatttaacttttttctttttccttttaaatctctaatacatttggaatttaattttcttactgattaaactttacttgtatatgtttatattttataatgataatattaaaccatttgtaatgcttaaattaaatattaaatggtcataacaccatttatttaaagcctattctttctttcaggtttgatatactgactttttcatcatgttgataatcttgtgatatattcctgttaatgctccattggtttctacattttttatcctgttcttataagttgtctgtctcctcctgtggctgttctattttattttaattactgaaactttattggacaatagtaagctcagaagagaatatctgtaggacaacagtaggctcaaggagaaatcatctgttctcctaatcttttactatttttcgtattttcctgactgatcttcagtatttctggaaaattgacaagcaatgcagattttggataatagtgcaggcttcactttcattgtgcctgggttcaaatcccagcttcttagctgactggcaatgtgacccagcaaatttatttactttggcatggctcagttttcttatctgtaaagtaagggtaatagtagtccctatattataagtttattatgatgattgtgtgggtggaacccgtgtgtggcacacaccggcacttagtgagaagttagctttgccattttcctttattcttcactctcgagcctaaaacttaaagtataataaaaaaataaataaataaataaaaaagaaaagaatgaactgaaagtgacaggttgccagagaggtggaaccacatggaatagagcagagaaagaaggagataaagatacagactacagcctattcagagtgacttacaaatgcaccgttctggataggatgccgtggggcagcctctcagcacacatatcagctaggctaattatatgttaattgtagttatttatcctggttttataatataatttctctggaagttgttagaatttttattctaactttaatgttctgaaatttcatatacatatggaataaattgctttggatttttatttgttttattttccatttgatttaatttggttttgtaaatcctgctcaacactgagtagggtcttttttctaatctcctaactttatttctctataaaggaatattctctgttatctcttaaaatattggcagctccgtattacgactaatcttttgaaactctatttagatgatttttgggaacttctgggtctgttctccaattctcttcccttttattatttctctgtctttagtttcagctttataattaattctactgccacatacattttgcactttggcctataaatttcacaatttgtgtttttttatttttatagttctagttggttcttctttatatccacctagtcatgttatggaataaaataccatggctccaaagacaaaaaagactgtctcttttattctttgcgtaacttaccagctccaactgtggcctgaacttctgctatttatcattgagaagtgagaagatatagaattagccccactgactgaacactagtttcctgtaatgctcttcattgctctccggcattcattaccagataaccaaggcatgtcccggagtatctaatgtaaactcacatattcactattcatcctggggcgaggtccatagatttctgctgcctgtccaaagaagcataggttgaactatcaaattgcttcaatgatccagggaattaattgctcttaaacctcctcctacttttctatactgcagaaatatcacatctaaagtaacaatatttcttgatcttttgtgctgcaattatcctcttcagcttctacttcctaatgactgctcagtttctatttaccaataattgctcataattgccataattctcttgcttttttaattttttaattttttatttttttgagacaaagtcttactctgtcgcccaagctgtagtgtagtggcgcgatctcagttcactgcaacttccacctcccgggttcaagcgattctactgcctcaacctcctgagtagctgggactaggtacctttactcaggagcttttgaacacagtagcattctagcagaaatgagccttgacaagttggttttttttttttttttttgaaatgtagtttcactcttgtcgcccaggctggagtgcaatggcgtgatgatctcagctcactgcaacatctgcctcccaggttcaagtgattctcccacgtcagcctcctgagtagctgggattacaggcacctgccaccatgcccagctaatttttgtatttttagtagcgatggggttttgacatgttgaccaggctggtctcaaactcctgacctcaggtgaaccgcccaccttggcctgaccaaagtgctgggattacaggcgtgagccatcatgccccgttgacatgtcggattctttaaagagaaattgtctctagacagcagaatatggactgcccactcagcccagatatctctcaataataataataataatagaagaagaaggagaaggaggaggaggagaaggagcagaaggagaaggagaaggagaagaaaggagaaggagaaggagaagaagaagaagaagaggaggaggaggaggaggaggaggaggacgaagaagaaaaggttctttaaagtgagaccttaaggaagttatgactgactgacccttactatgtagacccgcttttttttttggagacagtgtcttgctctgttgtccaggctggagtgcagtggcgtgatcatagctcactgcagcaatgaacttctgggcttaagtgatcctcctgccttaacctcctgaatagctaggactacgggtgcacaccaccatgcccagctaatttttttagttttatttttgtagagatgggggtctcactatgttcccccaggatggtcttgaactcctagcctcaaacaatcctttgatcctccctcctcagcctcccaaagggctgggattacaggcatgagctgctgctcccttagagacctgctttttcacataaattttaagttcctaaaacagactgtccggctcctcaggtagctcttaaccttgagttcctccagtcaccttgggctagctctggtccagccccagccctcttcagcagtgcccttgaccttgtctctcacccttgtctcctgcgttctagaggtcctgggctattcactcctggcttcaacccttccttacccttgactctggcttactcacaaccagtgctatccataaggagtgaatccctattgctttatttctacaaaggatcc

SEQ ID NO: 15 - DNA, 3796 bp, Homo sapiens
gaattctcttttgatgtataatattttaattagtttattgaaaatctctgtccttcctttttgaatttagtttatatcttttgcatattttaaagtttagattgttgtcttttctgttattaatttataaaatatttgtgttgagtgtgtgagtgcatgtgcatgcatgtgtatgcgtatgtgtgtgtttgtaacattttcatcagttttttgcttgccctctatttatggtattttcagatgtgattagatttatttggtctttgacattatgatttctgtctctatcattgagtttagaaggccaatattattcctacctcattaccaaagaaatacttaattctatctgcttacagatttaacttttttctttttccttttaaatctctaatacatttggaatttaattttcttactgattaaactttacttgtatatgtttatattttataatgataatattaaaccatttgtaatgcttaaattaaatattaaatggtcataacaccatttatttaaagcctattctttctttcaggtttgatatactgactttttcatcatgttgataatcttgtgatatattcctgttaatgctccattggtttctacattttttatcctgttcttataagttgtctgtctcctcctgtggctgttctattttattttaattactgaaactttattggacaatagtaagctcagaagagaatatctgtaggacaacagtaggctcaaggagaaatcatctgttctcctaatcttttactatttttcgtattttcctgactgatcttcagtatttctggaaaattgacaagcaatgcagattttggataatagtgcaggcttcactttcattgtgcctgggttcaaatcccagcttcttagctgactggcaatgtgacccagcaaatttatttactttggcatggctcagttttcttatctgtaaagtaagggtaatagtagtccctatattataagtttattatgatgattgtgtgggtggaacccgtgtgtggcacacaccggcacttagtgagaagttagctttgccattttcctttattcttcactctcgagtgacaggttgccagagaggtggaaccacatggaatagagcagagaaagaaggagataaagatacagactacagcctattcagagtgacttacaaatgcaccgttctggataggatgccgtggggcagcctctcagcacacatatcagctaggctaattatatgttaattgtagttatttatcctggttttataatataatttctctggaagttgttagaatttttattctaactttaatgttctgaaatttcatatacatatggaataaattgctttggatttttatttgttttattttccatttgatttaatttggttttgtaaatcctgctcaacactgagtagggtcttttttctaatctcctaactttatttctctataaaggaatattctctgttatctcttaaaatattggcagctccgtattacgactaatcttttgaaactctatttagatgatttttgggaacttctgggtctgttctccaattctcttcccttttattatttctctgtctttagtttcagctttataattaattctactgccacatacattttgcactttggcctataaatttcacaatttgtgtttttttatttttatagttctagttggttcttctttatatccacctagtcatgttatggaataaaataccatggaatagtgattcacaattttatcattgcttctacatttactggttgtcactctactgtaggaaataggtcctccacacctccatttactttgtttacttatttatgtatttattgatcagcagtttcacagatcattttaatttaaagatgatcatccattattcttattttgatgttcacattgtcctaggttaggccagtgggagccctttcaggctacctctttgctctttcactacttgcattttaaaagttttatgcataccttcctgccaaagttgtgacatttttaatatgaagaagtttttaattttgagaaaatctaattcattttttttgtggttttaatttctgtgtcctgagacacatttgcctacacacaagtcacaaagattttctcctatattttttatctaaaagctttatagttttgtttttatatttcagtctataatacatctcaaattaatttttgtgttttgtgtgaagtagggtctaggtatattgctttcccaaaggacagctagttttcctaacacgatttgttaagattattcattttcctaatgaatttttgaccaaacaaatcaaatggacatctaagtgtgtctctagtttttagccctttattctattactttaatctatttattggttctgtgccagtaaaataataatttgattacagcatattatagttaatcttgaggttatgtagtgtatgtctcccaagtttgttcatctttctcaagtttgctttggatagtctaggtccttaaggtacctttactcaggagcttttgaacacagtagcattctagcagaaatgagccttgacaagttggttttttttttttttttttgaaatgtagtttcactcttgtcgcccaggctggagtgcaatggcgtgatgatctcagctcactgcaacatctgcctcccaggttcaagtgattctcccacgtcagcctcctgagtagctgggattacaggcacctgccaccatgcccagctaatttttgtatttttagtagcgatggggttttgacatgttgaccaggctggtctcaaactcctgacctcaggtgaaccgcccaccttggcctgaccaaagtgctgggattacaggcgtgagccatcatgccccgttgacatgtcggattctttaaagagaaattgtctctagacagcagaatatggactgcccactcagcccagatatctctcaataataataataataatagaagaagaaggagaaggaggaggaggagaaggagcagaaggagaaggagaaggagaagaaaggagaaggagaaggagaagaagaagaagaagaggaggaggaggaggaggaggaggaggacgaagaagaaaaggttctttaaagtgagaccttaaggaagttatgactgactgacccttactatgtagacccgcttttttttttggagacagtgtcttgctctgttgtccaggctggagtgcagtggcgtgatcatagctcactgcagcaatgaacttctgggcttaagtgatcctcctgccttaacctcctgaatagctaggactacgggtgcacaccaccatgcccagctaatttttttagttttatttttgtagagatgggggtctcactatgttcccccaggatggtcttgaactcctagcctcaaacaatcctttgatcctccctcctcagcctcccaaagggctgggattacaggcatgagctgctgctcccttagagacctgctttttcacataaattttaagttcctaaaacagactgtccggctcctcaggtagctcttaaccttgagttcctccagtcaccttgggctagctctggtccagccccagccctcttcagcagtgcccttgaccttgtctctcacccttgtctcctgcgttctagaggtcctgggctattcactcctggcttcaacccttccttacccttgactctggcttactcacaaccagtgctatccataaggagtgaatccctattgctttatttctacaaaggatcc

SEQ ID NO: 16 - DNA, 62 bp, Artificial
  synthesized DNA
gatcacgcgtaggcctagaattcattctcgagtatggtacctcaggatcccggaccgagtac

SEQ ID NO: 17 - DNA, 54 bp, Artificial
  synthesized DNA
tcggtccgggatcctgaggtaccatactcgagaatgaattctaggcctacgcgt

SEQ ID NO: 18 - DNA, 29 bp, Artificial
  synthesized DNA
gaattctcttttgatgtataatattttaa

SEQ ID NO: 19 - DNA, 25 bp, Artificial
  synthesized DNA
ctcgagagtgaagaataaaggaaaa

SEQ ID NO: 20 - DNA, 24 bp, Artificial
  synthesized DNA
ggtacctttactcaggagcttttg

SEQ ID NO: 21 - DNA, 26 bp, Artificial
  synthesized DNA
ggatcctttgtagaaataaagcaata

SEQ ID NO: 22 - DNA, 23 bp, Artificial
  synthesized DNA
ctcgagtgacaggttgccagaga

SEQ ID NO: 23 - DNA, 22 bp, Artificial
  synthesized DNA
gcagaagttcaggccacagttg

SEQ ID NO: 24 - DNA, 30 bp, Artificial
  synthesized DNA
ccatggaatagtgattcacaattttatcat

SEQ ID NO: 25 - DNA, 30 bp, Artificial
  synthesized DNA
ggtaccttaaggacctagactatccaaagc

SEQ ID NO: 26 - DNA, 19 bp, Artificial
  synthesized DNA
tgacagcagtgttgataaa

SEQ ID NO: 27 - DNA, 19 bp, Artificial
  synthesized DNA
tgacgaagtcgtgattaaa

SEQ ID NO: 28 - DNA, 19 bp, Artificial
  synthesized DNA
ggagtattgtggaacttat

SEQ ID NO: 29 - DNA, 19 bp, Artificial
  synthesized DNA
ggagtagtcgtaatgttat

(19) United States
(12) Patent Application Publication
(10) Pub. No.: US 2010/0263066 A1
(43) Pub. Date: Oct. 14, 2010

(54) INERT DNA SEQUENCES FOR EFFICIENT VIRAL PACKAGING AND METHODS OF USE

(75) Inventors: Michael D. Kaytor, Maplewood, MN (US); Jennifer M. Heisel, Princeton, MN (US); Eric Neal Burright, Eagan, MN (US); Jocelyn Clark-Gruel, Anoka, MN (US)

Correspondence Address: Kenneth J. Collier - c/o Kathy Illg;Medtronic, Inc., 710 Medtronic Parkway NE, Mail Stop LC-340, Minneapolis, MN, 55432, US

(73) Assignee: Medtronic, Inc, Minneapolis, MN (US)

(21) Appl. No.: 12/598,333

(22) PCT Filed: Apr. 30, 2008
(86) PCT No.: PCT/US08/62013

§ 371 Date: May 25, 2010

Related U.S. Application Data
(60) Provisional application No. 60/915,071, filed on Apr. 30, 2007.

(51) Int. Cl. A01K 67/027; C07H 21/04; C12N 15/63; A61K 31/713; G01N 33/48; C12N 5/07; A61P 43/00; A61M 5/32

(52) U.S. Cl. 800/14; 536/23.1; 435/320.1; 514/44 A; 436/94; 435/325; 435/366; 604/175

(57) ABSTRACT

The instant invention provides an inert DNA sequence having a length of between about 0.5 kb and about 5 kb, wherein said isolated inert DNA sequence does not contain an open reading frame and which is suitable for efficient packaging of expression cassettes comprising a nucleic sequence encoding a therapeutic agent into viral vectors, as well as methods of selecting such inert DNA sequences. The invention also provides DNA constructs and medical composition comprising such inert DNA sequences, and kits and medical systems for delivering such DNA constructs and/or compositions.

### CROSS-REFERENCE TO RELATED APPLICATIONS

This application claims the benefit of U.S. Provisional Application Ser. No. 60/915,071 filed on Apr. 30, 2007, which is incorporated herein by reference.

### FIELD OF THE INVENTION

The present invention is directed to an inert DNA sequence having a length of between about 0.5 kb and about 5 kb, wherein said isolated inert DNA sequence does not contain an open reading frame and which is suitable for efficient packaging of expression cassettes comprising a nucleic sequence encoding a therapeutic agent into viral vectors, as well as methods of selecting such inert DNA sequences.

### BACKGROUND OF THE INVENTION

Gene therapy using RNA interference is a rapidly expanding field. However, the delivery of the RNA interference agents is still a major problem. Currently, one of the most promising methods to deliver therapeutic short interfering nucleic acids (siNAs) or short hairpin nucleic acids (shNAs) entails packaging these siRNAs or shRNAs into viral particles. Multiple viral vectors have been investigated in delivering gene therapy to a patient.

For example, adeno-associated virus (AAV) is widely used to deliver molecular therapies for the treatment of clinical disorders. Wild-type AAV has a genome size of 4.7 kb. To maintain efficient viral packaging the genome should be between 3.5, and 4.7 kb. Currently, most AAV-based treatments are gene replacement therapies in which a gene is delivered to replace a defective or absent gene. Because most genes can be engineered to be about 4 kb in length, viral packaging is not an issue. If the viral genome size is smaller, 2.3 kb or less, multimers of the genome may be packaged to attain the optimal size of 4.7 kb. This is problematic if gene dosage is a concern for the therapy. This is particularly the case in AAV-mediated RNA interference therapies. In this therapy the cassette to express the short hairpin nucleic acid (shNA) is about 1 kb in length. Typically, to reach the optimal AAV genome size (4.7 kb), a reporter gene such as green fluorescent protein (GFP) is included. However, in an AAV vector to be used in human clinical trials, GFP is not desirable. Secondly, due to possible dose-dependent toxicity, the expression of multiple copies of the shNA is not an option.

Therefore, there is a need in the art for inert DNA sequences suitable for adjusting the length of therapeutic DNA constructs for efficient viral packaging.

### SUMMARY OF INVENTION

The instant invention addresses this and other needs of the prior art by providing, in one aspect, an isolated inert DNA sequence having a length of between about 0.5 kb and about 5 kb, wherein said isolated inert DNA sequence contains no open reading frame. In different embodiments, the isolated inert DNA of the instant invention may also possess one or more of the following characteristics: a) it does not contain a polII promoter and preferably does not contain a polIII promoter; b) it contains no CpG islands; c) it does not contain a splice donor site or a splice acceptor site; d) it does not contain an miRNA sequence; e) it does not comprise a portion of an imprinting center; f) it does not comprises a functional histone binding site. In further embodiments, the isolated inert DNA of the instant invention is at least 75% identical to a portion of a genome of a mammal of family Hominidae or family Cercopithecidae, including, without limitations, Homo sapiens and Macaca mulatto. In specific embodiments, the isolated inert DNA sequence of the instant invention comprises SEQ ID Nos: 1-15, which are recited in the 5′ to 3′ orientation.

In a second aspect, the invention provides a DNA construct comprising: a) a first part comprising a nucleic acid sequence encoding a bioactive nucleic acid; and b) the isolated inert DNA sequence according to any one of the previously described embodiments and having length of between about 0.5 kb and about 4.5 kb. In an additional embodiment, the DNA construct further comprises a part of viral genome. Preferably, in this embodiment, the length of such DNA construct is suitable for packaging. In one embodiment, the virus is an adeno-associated virus, and the length of the DNA construct is between about 3.5 and about 5 kb. In one set of embodiments, the bioactive nucleic acid is a short interfering nucleic acid (siNA) or short hairpin nucleic acid (shNA). In a preferred embodiment, the bioactive acid is a short interfering RNA (siRNA) or short hairpin RNA (shRNA).

In a third aspect, the invention provides a composition comprising the DNA construct of any one the embodiments described above packaged into a viral capsid. In different embodiments, the composition may further comprise a carrier or diluent. In another embodiment, the composition is in a sustained-release formulation.

In a fourth aspect, the invention provides a method of selective inhibition of a target gene in a live mammal comprising administering to said mammal the DNA construct of any embodiment of the second aspect of the invention or the composition of any, embodiment of the third aspect of the invention, or a combination thereof, wherein the bioactive nucleic acid cleaves the target mRNA via RNA interference.

In a fifth aspect, the invention provides a method of selecting inert DNA sequences in a genome comprising an identification of target sequences having length above 0.5 kb and containing no open reading frame. In different embodiments, the method may also comprise one or more of the following steps: a) selecting the target sequences containing no polII and preferably no polIII promoter; b) selecting the target sequences containing no CpG islands; c) selecting the target sequences that do not contain a splice donor site or a splice acceptor site; d) selecting the target sequences that do not contain miRNA or miRNA precursor sequences; e) selecting the target sequences having the greatest intra-species variation; e) selecting the target sequences not comprising a functional histone binding site; f) selecting the target sequence not comprising a portion of an imprinting center. In one embodiment of the invention, the mammal is human.

In a sixth aspect, the invention provides a medical system comprising: a) an intracranial access port; b) mapping means for locating a pre-determined target area in a brain of a patient, said pre-determined target area comprising cells natively expressing a gene involved in a neurodegenerative disorder; c) at least one of the DNA construct according or the composition according to any one of the embodiments described in the instant disclosure; and d) a delivery means.

In a seventh aspect, the invention provides a kit comprising: a) at least one of the DNA construct or the composition according to any one of the embodiments described in the instant disclosure; and b) a set of instructions. In a particular embodiment, the kit further comprises any one of: c) an intracranial access port; d) mapping means for locating a pre-determined target area in a brain of a patient, said predetermined target area comprising cells natively expressing a gene involved in a neurodegenerative disorder; and, e) a delivery means.

In an eighth aspect, the invention provides a mammalian cell comprising at least one of the DNA construct or the composition according to any one of the embodiments described in the instant disclosure.

In a ninth aspect, the invention provides a non-human mammal comprising at least one of the DNA construct or the composition according to any one of the embodiments described in the instant disclosure.

### BRIEF DESCRIPTION OF THE DRAWINGS

FIG. 1 is a schematic map of pAAV plasmids, used in Example 2. ITR refers to inverted terminal repeat, U6-shRNA refers to human U6 promoter driving expression of shRNAs directed against Huntington or scrambled inactive versions thereof, and inert DNA refers to inert DNA sequences according to any embodiment of this application. In addition, these pAAV plasmids have been used to generate AAV1 by the 3-plasmid system. The generation of rAAV1 by this method includes the use of a plasmid expressing the rep and cap genes and a plasmid expressing helper virus functions.

FIG. 2 illustrates the specific suppression of endogenous human HD gene expression by HD-1 and HD-5 shNA containing plasmid constructs measured by realtime PCR and normalized to the amount of endogenous human GAPDH expressed.

FIG. 3 is a schematic illustration of AAV genome.

FIG. 4 illustrates that the addition of the inert DNA sequence does not affect the specificity of rAAV mediated attenuation of IT15 (huntingtin) mRNA by shRNA as measured by real time PCR and normalized to the amount of endogenous human GAPDH.

FIG. 5 illustrates the in vitro validation of rAAV serotype 1 expressing shNA against HD and containing INERT DNA sequence (INERTverC).

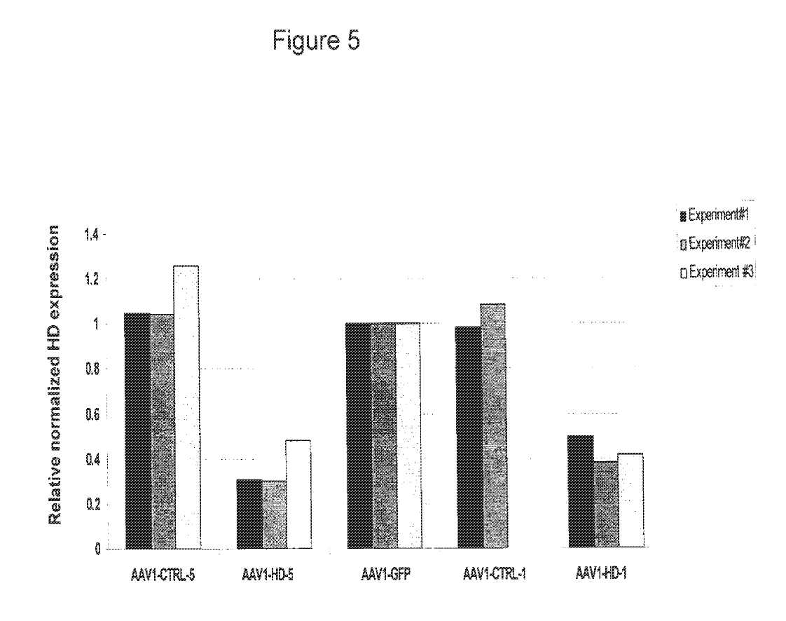

FIG. 6 illustrates the in vitro validation of rAAV serotype 2 expressing shNA against HD and containing INERT DNA sequence (INERTverC).

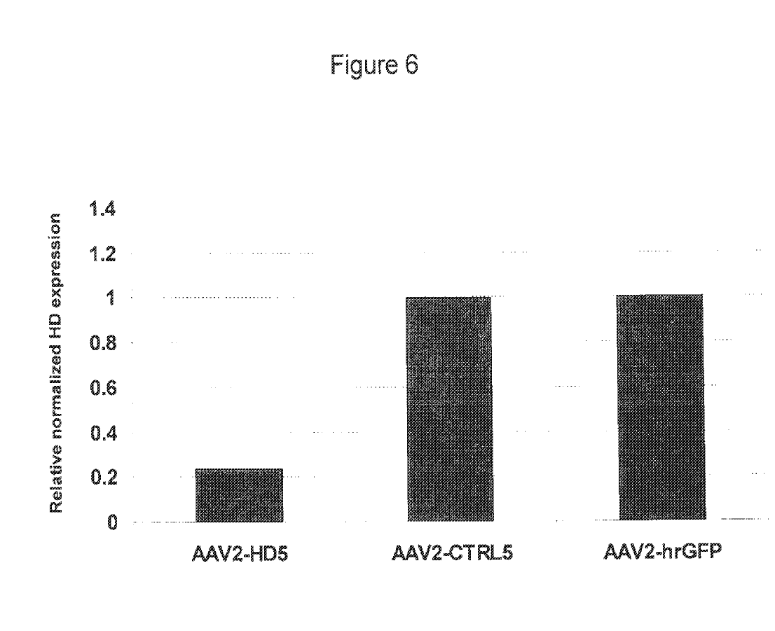

FIG. 7 illustrates Huntington mRNA suppression in rhesus monkey (AAV2-HD5)

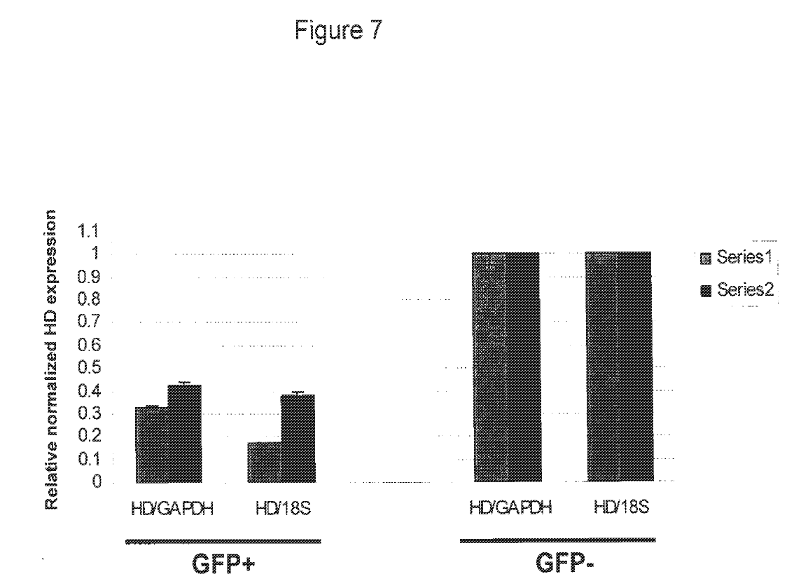

FIG. 8 illustrates the effects of long-term expression of rAAV serotype 2 containing INERT DNA (INERTverC) in mice.

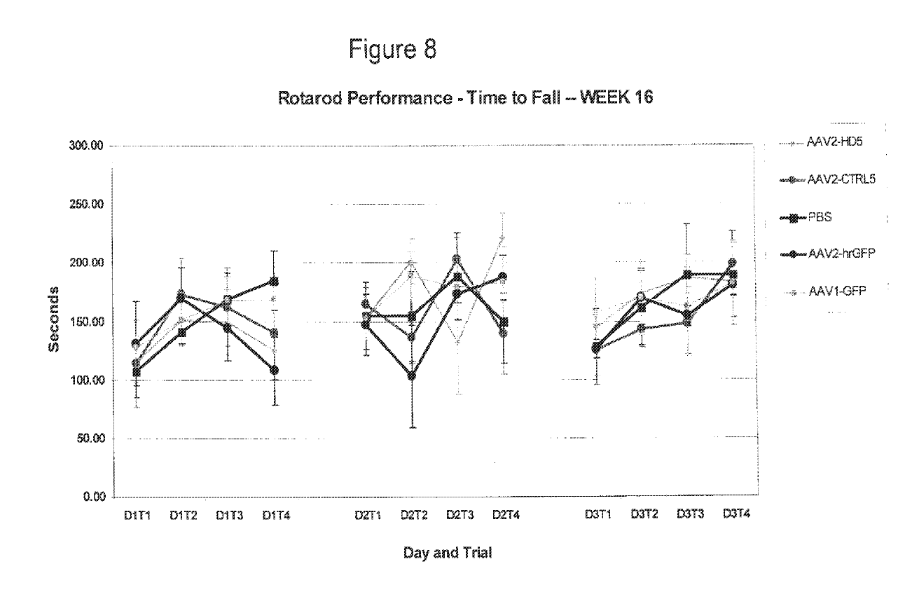

FIG. 9 illustrates the inclusion of INERT DNA on a plasmid or within a rAAV viral genome does not interfere with the expression of the red fluorescent reporter gene.

### DETAILED DESCRIPTION OF PREFERRED EMBODIMENTS

For the purposes of a better description of the preferred embodiments of the instant invention, the following non-limiting definitions shall apply:

### Definitions

The term “inert” refers to DNA sequences that are not transcribed into mRNA or parts thereof (e.g., introns).

The DNA sequence “does not contain an open reading frame” if the analysis of this sequence with the online analysis tool on the NCBI web site (http://www.ncbi.nlm.nih.gov/gorf/gorf.html) predicts no standard open reading frames (beginning with methionine), wherein the threshold is at 130 base pairs or greater.

The inert DNA sequence “does not contain polII promoter” if analysis of this DNA sequence using Neural Network Promoter Prediction software (available at http://www.fruitfly.org/seq_tools/promoter.html, the Berkeley Drosophila Genome Project website) predicts that the analyzed sequence contains no promoter, wherein “Position” is a position in the sequence, and the score is a measurement of likelihood of the promoter, and wherein the ‘Score’ is the prediction score for a transcription start site occurring within 100 base pairs upstream from that position and the cut-off score is equal to 1.

The accuracy of the software has been tested on a set of 100 vertebrate promoters. The positions scoring 0.5-0.8 (Marginal predictions) contain about 65% true transcription start sites within 100 base pairs upstream. The positions scoring 0.8-1.0 (Medium likely predictions) are about 80% true. Finally, the positions scoring above 1.0 (Highly likely predictions) are about 95% true. On average, the software picks up about 80% of all PolII promoters. These numbers are rough estimates based on a limited test set. A person of ordinary skill in the art will appreciate that the Neural Network Promoter Prediction software is a state-of-the-art tool for determining the occurrence of the promoter. See, e.g., Fickett and Hatzigeorgiou, Genome Res. 7(9), 861-878, 1997.

The DNA sequence “contains no CpG islands” or “does not contain a CpG island” if:
1) CpG Island Searcher software (available at http://www.uscnorris.com/cpgislands2/cpg.aspx, the University of Southern California/Norris Comprehensive Cancer Center website) predicts that the sequence contains no CpG islands, assuming the following parameters: a) lower cut-off value for % GC is 50%; b) lower cut-off value for OBSCpG/EXPCpG is 0.6; c) lower cut-off value for length is 200 bp; and d) lower cut-off value for gap between adjacent islands is 100 bp; and
2) EMBOSS CpGPlot/CpGReport/Isochore software (available at http://www.ebi.ac.uk/emboss/cpgplot/, the European Bioinformatics Institute website) predicts that the sequence contains no CpG islands, assuming the following parameters: a) MinPC is equal to 50; b) Step is equal to 1; c) ObsCpG/ExpCpG is equal to 0.6; d) Minimal Length is equal to 200 bp; e) Window is equal to 100; f) both reverse sequence and complement strand is searched. In this analysis, “WINDOW” refers to a window having size set by this parameter. The window is moved down the sequence and these statistics are calculated at each position that the window is moved to; “STEP” determines the number of bases that the window is moved each time after values of the percentage CG content and the observed frequency of CG are calculated within the window; “OBS/EXP” sets the minimum average observed to expected ratio of C plus G to CpG in a set of 10 windows that are required before a CpG island is reported; “MINPC” sets the minimum average percentage of G plus C a set of 10 windows that are required before a CpG island is reported; “LENGTH” sets the minimum length that a CpG island has to be before it is reported; “REVERSE” allows the researcher to reverse the sequence being analyzed; and “COMPLEMENT” allows the researcher to reverse/complement the sequence being analyzed.

The DNA sequence “does not contain a splice donor site or a splice acceptor site” if the analysis of the DNA sequence with NetGene2 Server software (available at http://www.cbs.dtu.dk/services/NetGene2, the website for the Center for Biological Sequence Analysis, Technical University of Denmark DTU) predicts that the analyzed DNA sequence does not contain a splice donor site or a splice acceptor site with a confidence level (probability that the revealed site is a true splice donor or acceptor cite) of 75% or more. For a detailed explanation, see S. M. Hebsgaard, P. G. Korning, N. Tolstrup, J. Engelbrecht, P. Rouze, and S. Brunak, “Splice site prediction in Arabidopsis thaliana DNA by combining local and global sequence information,” Nucleic Acids Research, 24(17): 3439-3452 (1996); S. Brunak, J. Engelbrecht, and S. Knudsen, “Prediction of Human mRNA Donor and Acceptor Sites from the DNA Sequence,” Journal of Molecular Biology, 220: 49-65 (1991).

The DNA sequence “does not contain a miRNA sequence” is a conclusion of a two-step analysis. First, analysis is performed with miRBase::Sequence software (available at the Wellcome Trust Sanger Institute website, http://microrna.sanger.ac.uk/sequences/search.shtml,) wherein the search is performed for mature miRNA and stem loop sequence by BLASTN method with E-value threshold of 10. If the miRNA precursor sequence is found after the analysis described above, a second step of the analysis is performed. The second step involves MFOLD version 3.2 analysis with MFOLD software (by Zuker and Turner, Rensselaer Polytechnic Institute; available at the website for the Bioinformatics Center at Rensselaer and Wadswort) available from http://www.bioinfo.rpi.edu/applications/mfold/cgi-bin/rna-form1.cgi, with the following parameters: a) the RNA sequence is linear; b) folding temperature is fixed at 37° C.; c) ionic conditions are set at 1M NaCl with no divalent ions; d) the percent suboptimality number is equal to 5; e) an upper bound on the number of computed foldings is equal to 50; f) the window parameter is set at default; g) the maximum interior/bulge loop size is equal to 30; h) the maximum asymmetry of an interior/bulge loop is equal to 30; i) the maximum distance between paired bases is set at “no limit”. See Zuker, Nucleic Acids Res. 31(13): 3406-15, (2003); Mathews et al., J. Mol. Biol. 288: 911-940 (1999).

The term “promoter element” or “promoter” or “regulatory region” refers to a DNA regulatory region capable of being bound by an RNA polymerase in a cell (e.g., directly or through other promoter-bound proteins or substances) and allowing for the initiation of transcription of a coding or non-coding RNA sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The term “in operable combination,” “in operable order,” or “operably linked” refers to the linkage of nucleic acid sequences in such a manner that a first nucleic acid molecule (e.g., promoter) is capable of directing the transcription of a second nucleic acid sequence (e.g., siNA or shNA).

The term “vector” refers to a nucleic acid assembly capable of transferring gene sequences to target cells (e.g., viral vectors). The term “expression vector” refers to a nucleic acid assembly containing a promoter that is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells. Thus, the term includes cloning and expression vehicles, as well as viral vectors.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing as well as after their appearance. Thus, “treating” or “treatment” includes “preventing” or “prevention” of disease. In addition, “treating” or “treatment” does not require complete alleviation of signs or symptoms, does not require a cure, and specifically includes protocols that have only a marginal effect on the patient.

The term “patient” refers to a biological system to which a treatment can be administered. A biological system can include, for example, an individual cell, a set of cells (e.g., a cell culture), an organ, a tissue, or a multi-cellular organism. A “patient” can refer to a human patient or a non-human patient.

The term “practitioner” refers to a person who uses methods, kits, and compositions of the current invention on the patient. The term includes, without limitations, doctors, nurses, scientists, and other medical or scientific personnel.

The terms “miRNA molecule,” “siRNA molecule,” “shRNA molecule,” “RNA molecule,” “DNA molecule,” “cDNA molecule” and “nucleic acid molecule” are each intended to cover a single molecule, a plurality of molecules of a single species, and a plurality of molecules of different species.

The term “siNA” is intended to cover siRNA as well as siDNA sequences. The term “shNA” is intended to cover shRNA as well as shDNA sequences.

The term “AAV rep coding region” refers to the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N., Current Topics in Microbiol. and Immunol., 158: 97-129 (1992); Kotin, R. M., Human Gene Therapy 5: 793-801 (1994). Suitable homologues of the AAV rep coding region include the human herpesvirus 6 (HHV-6) rep gene which is also known to mediate AAV-2 DNA replication (Thomson et al., Virology 204: 304-11 (1994)).

The term “AAV cap coding region” refers to the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

Thus, in the first aspect, the invention provides an isolated inert DNA sequence having a length of between about 0.5 kb and about 5 kb, wherein said isolated inert DNA sequence contains no open reading frame. In different embodiments, the isolated inert DNA of the instant invention may also possess one or more of the following characteristics: a) it does not contain a polII promoter and preferably does not contain a polIII promoter; b) it contains no CpG islands; c) it does not contain a splice donor site or a splice acceptor site; d) it does not contain a miRNA sequence; e) it does not comprise a functional imprinting center; f) it does not comprise a functional histone binding site.

In further embodiments, the isolated inert DNA of the instant invention is at least 75% identical to a portion of a genome of a mammal of family Hominidae or family Cercopithecidae, including, without limitations, Homo sapiens and Macaca mulatta. In specific embodiments, the isolated inert DNA sequence of the instant invention comprises SEQ. ID NOs 1-12.

In an additional set of embodiments, the isolated inert DNA of the invention is derived from a genome of a mammal of family Hominidae or family Cercopithecidae, and including, without limitations, Homo sapiens and Macaca mulatta. For example, the practitioner may select several short inert DNA sequences and assemble them into a long one. Suitable examples of these sequences are INERTverA, INERTverB, and INERTverC, designated as SEQ ID NOs 13-15, respectively.

In general, the selection process is self-explanatory in view of the information contained in the “definitions” subsections. A person of ordinary skill in the art would further appreciate that Human Genome Resources database (available at the website for the National Center for Biotechnology Information, National Institutes of Health, http://www.ncbi.nlm.nih.gov/projects/genome/guide/human/) permits sequence searching. To increase the efficiency of the search, it is preferable to search for large regions of sequence that contain no known genes. It is further preferable to avoid regions that contain human EST or other mRNA. A table listing the ESTs and mRNAs in order for a given chromosome will be suitable for this purpose. A person of the ordinary skill in the art will undoubtedly appreciate that it is not necessary that the inert DNA sequence of the instant invention should have all criteria described above. Notably, the greater the number of characteristics described above that are present, the more preferable the embodiment. The most crucial characteristics of the inert DNA sequence of the present invention are that this inert DNA sequence should not be transcribed and that it should not contain a long (e.g., over 130 bp) ORF. The inert DNA sequence may be purified from genomic DNA which may easily be obtained from blood, e.g., from a volunteer blood, from the patient's blood, or from cultured cell lines, e.g., human cell lines, such as, for example, HEK293T cell line. The inert DNA sequence within the genomic DNA may be multiplied by PCR, with several rounds of PCR if necessary. General primer selection criteria and PCR conditions are described, for example, in Ausubel et al., Current Protocols in Molecular Biology, 5th Edition, 2002, and may be optimized without undue experimentation.

The primers may further be modified by addition of endonuclease restriction sites for easier cloning of the inert DNA sequence into the therapeutic DNA construct, which may or may not include at least a portion of viral genome. After a person of the ordinary skill in the art multiplied the desired sequence by DNA, this sequence may be verified by restriction mapping or sequencing. Both of these procedures are known in the art extremely well and do not need any additional explanation.

### Constructs

In another aspect, the invention provides a DNA construct comprising a first part, which contains a nucleic acid sequence encoding a bioactive nucleic acid sequence, and a second part which contains at least a portion of the inert DNA sequence according to any embodiment described above. A person of ordinary skill in the art will undoubtedly appreciate that relative positions of the first part and the second part of the DNA construct is not essential. For example, in one embodiment, the first part is located upstream of the second part. In another example, the first part is located downstream of the second part. In a third embodiment, the first part is located inside the second part (i.e., the portions of the second part are present both upstream and downstream of the first part).

As discussed in details below, in different embodiments of the invention, the second part may comprise only one inert DNA sequence, or several (e.g., 2, 3, 4, etc.) inert DNA sequences, or only a portion of one inert DNA sequence, or a combination of one or more full inert DNA sequences and one or more portions of these inert DNA sequences. Thus, the term “at least a portion” should be interpreted to describe all these embodiments. In different embodiments, the portion of the inert DNA sequence may be as short as, for example, at least about 0.1 kb, or may be a longer sequence (e.g., at least about 0.2 kb, at least about 0.3 kb, etc). Suitable non-limiting examples of such portions include fragments from 2_I 1285 (SEQ ID NO 4), 3_H 1990 (SEQ ID NO 7), 3_I 1408 (SEQ ID NO 9), and 2_F 1245 (SEQ ID NO 12), as described in the Examples, and suitable combinations of these fragments include INERTverA, INERTverB, and INERTverC, (SEQ ID NOs 13-15, respectively).

In one embodiment, the bioactive nucleic acid sequence is an RNA interference agent, including, without limitations, siNA or shNA. The design and use of siNA molecules complementary to mRNA targets that produce particular proteins is a recent tool employed by molecular biologists to prevent translation of specific mRNAs. Various groups have been recently studying the effectiveness of siRNAs as biologically active agents for suppressing the expression of specific proteins involved in neurological disorders. For example, Caplen et al. (Human Molecular Genetics, 11(2): 175-184 (2002)) assessed a variety of different double stranded RNAs for their ability to inhibit cell expression of mRNA transcripts of the human androgen receptor gene containing different CAG repeat lengths. They were also able to show that constructed double stranded RNAs were able to rescue caspase-3 activation induced by expression of a protein with an expanded polyglutamine region. Xia, Mao, et al., Nature Biotechnology, 20: 1006-1010 (2002), demonstrated the inhibition of polyglutamine (CAG) expression in engineered neural PC12 clonal cell lines that express a fused polyglutamine-fluorescent protein using constructed recombinant adenovirus expressing shRNAs targeting the mRNA encoding green fluorescent protein.

Thus, one aspect of the present invention provides an siNA molecule corresponding to at least a portion of a target gene. In different embodiments, the siNA molecule comprises or encodes siRNA or shRNA. siRNAs are typically short (19-29 nucleotides), double-stranded RNA molecules that cause sequence-specific degradation of complementary target mRNA in a process known as RNA interference (RNAi) (Bass, Nature 411:428 (2001)).

Accordingly, in some embodiments, the siNA molecules comprise a double-stranded structure comprising a sense strand and an antisense strand, wherein the antisense strand comprises a nucleotide sequence that is complementary to at least a portion of a desired nucleic acid sequence and the sense strand comprises a nucleotide sequence that is complementary to at least a portion of the nucleotide sequence of said antisense region, and wherein the sense strand and the antisense strand each comprise about 19-29 nucleotides.

Any desired nucleic acid sequence can be targeted by the siNA molecules of the present invention. Nucleic acid sequences encoding desired gene targets are publicly available from Genbank. In one embodiment an siNA molecule corresponds to at least a portion of a gene containing an SNP variant of an allele in a heterozygous subject that is on the same mRNA transcript as a disease-causing mutation located at a remote region of the gene's mRNA, wherein such siRNA nucleic acid sequence is capable of inhibiting translation of the mRNA for the allele containing the disease-causing mutation in a cell. This embodiment is particularly suitable for allele-specific therapy as described below.

The siNA molecules targeted to desired sequence can be designed based on criteria well known in the art (e.g., Elbashir et al., EMBO J. 20:6877 (2001)). For example, the target segment of the target mRNA preferably should begin with AA (most preferred), TA, GA, or CA; the GC ratio of the siRNA molecule preferably should be 45-55%; the siNA molecule preferably should not contain three of the same nucleotides in a row; the siNA molecule preferably should not contain seven mixed G/Cs in a row; the siNA molecule preferably should comprise two nucleotide overhangs (preferably TT) at each 3′ terminus; the target segment preferably should be in the ORF region of the target mRNA and preferably should be at least 75 by after the initiation ATG and at least 75 by before the stop codon; and the target segment preferably should not contain more than 16-17 contiguous base pairs of homology to other coding sequences.

Based on some or all of these criteria, siNA molecules targeted to desired sequences can be designed by one of skill in the art using the aforementioned criteria or other known criteria (e.g., Gilmore et al., J. Drug Targeting 12:315 (2004); Reynolds et al., Nature Biotechnol. 22:326 (2004); Ui-Tei et al., Nucleic Acids Res. 32:936 (2004)). Such criteria are available in various web-based program formats useful for designing and optimizing siRNA molecules (e.g., siDESIGN Center at Dharmacon; BLOCK-iT RNAi Designer at Invitrogen; siRNA Selector at Wistar Institute; siRNA Selection Program at Whitehead Institute; siRNA Design at Integrated DNA Technologies; siRNA Target Finder at Ambion; and siRNA Target Finder at Genscript).

Short hairpin RNA (shRNA) molecules fold back on themselves to produce the requisite double-stranded portion (Yu et al., Proc. Natl. Acad. Sci. USA 99:6047 (2002)). Such RNA molecules can be produced using DNA templates (e.g., Yu et al., Proc. Natl. Acad. Sci. USA 99:6047 (2002)). Thus, shRNA molecules are essentially siRNA molecules wherein the first and the second strands are connected by a loop.

These siRNA and shRNA molecules may target genes involved in pathogenesis of multiple diseases, involving, without limitations BACE (e.g., BACE-1, BACE-2, BACE-3 and BACE-4) which is involved in Alzheimer's disease, SCA-1, involved in spinocerebellar ataxia 1, IT15 (also known as huntingtin) involved in Huntington's disease, and alpha-synuclein involved in Parkinson's disease. A more detailed unlimited list of the suitable diseases is shown in Table 1.

TABLE 1

| | | | | |
|---|---|---|---|---|
| Disease | Symptoms | Gene | Locus | Protein |

Non-coding repeats

| | | | | |
|---|---|---|---|---|
| Dystrophia | Weakness, | DMPK | 19q13 | Dystrophia |
| myotonica 1 | Myotonia | | | myotonica |
| | | | | Protein kinase |
| Spinocerebellar | Ataxia | Antisense | 13q21 | Undetermined |
| ataxia 8 | | to KLHL1 | | |
| Huntington disease- | Chorea, dementia | JPH3 | 16q24.3 | Junctophilin 3 |
| like2 | | | | |

Polyglutamine disorders

| | | | | |
|---|---|---|---|---|
| Spinal and bulbar | Weakness | AR | Xq13-q21 | Androgen |
| muscular atrophy | | | | receptor |
| Huntington disease | Chorea, dementia | IT15 | 4P16.3 | Huntingtin |
| Dentatorubral- | Ataxia, myoclonic | DRPLA | 12p13.31 | Atrophin 1 |
| pallidoluysian | epilepsy, | | | |
| atrophy | dementia | | | |
| Spinocerebellar | Ataxia | SCA1 | 6p23 | Ataxin 1 |
| ataxia 1 | | | | |
| Spinocerebellar | Ataxia | SCA2 | 12q24.1 | Ataxin 2 |
| ataxia 2 | | | | |
| Spinocerebellar | Ataxia | SCA3/MJD | 14q32.1 | Ataxin 3 |
| ataxia 3 (Machado- | | | | |
| Joseph disease) | | | | |
| Spinocerebellar | Ataxia | CACNA1A | 19p13 | α_{1A}-voltage- |
| ataxia 6 | | | | dependent |
| | | | | calcium |
| | | | | channel |
| | | | | subunit |
| Spinocerebellar | Ataxia | SCA7 | 3p12-p13 | Ataxin 7 |
| ataxia 7 | | | | |
| Spinocerebellar | Ataxia | TBP | 6q27 | TATA box |
| ataxia 17 | | | | binding |
| | | | | protein |

Polyalanine disorders*

| | | | | |
|---|---|---|---|---|
| Oculopharyngeal | Weakens | PABPN1 | 14q11.2-q13 | Poly(A)- |
| dystrophy | | | | binding |
| | | | | protein 2 |
| Congenital central | Respiratory | PHOX2B | 4p12 | Paired-like |
| hypoventilation | difficulties | | | homeobox 2B |
| syndrome | | | | |
| Infantile spasms | Mental | ARX | Xp22.13 | Aristaless- |
| | retardation, | | | related |
| | epilepsy | | | homeobox, X- |
| | | | | linked |
| Synpolydactyly | Limb malformation | HOXD13 | 2q31-q32 | Homeobox D13 |

*Polyalanine expansions have also been reported among mutations in other genes, including RUNX2 (runt-related transcription factor 2) in cleidocranial dysplasia, ZIC2 (Zic family member 2) in holoprosencephaly HOXA13 (homeobox A13) in hand-foot-genital syndrome, and FOXL2 (forkhead box L2) in type II blepharophimosis, ptosis, and epicanthus inversus syndrome. Small aspartic acid repeat expansions have been reported among other mutations in the COMP (cartilage oligomeric matrix protein) gene in patients with multiple epiphyseal dysplasia.

Multiple siRNA or shRNA molecules may be used to target their respective targets. Non-limiting examples of such sequences are HD-5 and HD-1, as described in the Examples.

In one embodiment, the nucleic acid sequences encoding the siRNA or shRNA molecules are included into an expression cassette. Generally, the expression cassette comprises a DNA sequence encoding the bioactive nucleic acid sequence (e.g., siRNA or shRNA), a regulatory sequence to direct the synthesis of such siRNA or shRNA, and a 3′ untranslated region (e.g., polyA site). Alternatively, if transcription occurs from a pol III promoter, termination occurs within a stretch of six thymine residues within the template DNA sequence.

The regulatory sequences may comprise a basic promoter, such as, for example, a TATA box within 20-30 bases from the start of transcription of the bioactive molecule. In other embodiments, the regulatory sequence may further comprise additional sequences which increase the expression of the bioactive nucleic acid. In one embodiment, the regulatory sequence may comprise highly active constitutively active promoters. Suitable eukaryotic promoters include constitutive RNA polymerase II promoters (e.g., cytomegalovirus (CMV) promoter, the SV40 early promoter region, the promoter contained in the 3′ long terminal repeat of Rous sarcoma virus (RSV), the herpes thymidine kinase (TK) promoter, and the chicken beta-actin promoter), and RNA polymerase III promoters (e.g., U6, H1, 7SK and 7SL). The person of ordinary skill will recognize that the sequences of these promoters are well known in the art.

As discussed above, multiple viral vectors have been investigated and found effective for delivery of gene, therapy agents to tissues. These viruses include, without limitations adenoviral vectors, adeno-associated viral vectors, lentiviral vectors, herpes simplex virus (HSV) poliovirus, feline immunodeficiency virus, and murine Maloney-based viral vectors. In one exemplary embodiment, the vector comprises an adeno-associated virus (AAV), from the parvovirus family. A person of ordinary skill in the art will recognize that among the advantages of AAV are the facts that AAV is not pathogenic and that most people treated with AAV will not build an immune response to remove the virus.

Both adenoviral and AAV vectors have been shown to be effective at delivering transgenes (including transgenes directed to desired target genes) into central nervous system cells. See, e.g., Bankiewicz et al., “Long-Term Clinical Improvement in MPTP-Lesioned Primates after Gene Therapy with AAV-hAADC”, Mol. Ther., E-publication Jul. 6, 2006 (A combination of intrastriatal AAV containing a nucleic sequence encoding L-amino acid decarboxylase inhibitor (AAV-hAADC) gene therapy and administration of the dopamine precursor 1-Dopa to MPTP-lesioned monkeys, resulted in long-term improvement in clinical rating scores, significantly lowered 1-Dopa requirements, and a reduction in 1-Dopa-induced side effects.); Machida et al., Biochem Biophys Res Commun. 343(1):190-7 (2006) (Reporting a direct inhibition of mutant gene expression by rAAV-mediated delivery of RNAi into the HD model mouse striatum after the onset of disease); Mittoux et al., J. Neurosci. 22(11):4478-86 (2002). (Adenovirus-mediated ciliary neurotrophic factor delivery to brain resulted in increased survival of striatal neurons in response to a neurotoxin).

A person of the ordinary skill in the art will appreciate that brain is not the only target area to which the systems, methods and kits of the instant invention can be administered. Another non-limiting example of the target area suitable to be treated with the systems, methods and kits of the instant invention is myocardium. In such embodiment, the target genes include, without limitation, phospholamban, SERCA2a, Kir2.1, KCNJ2, HCN2, and HCN4.

For purposes of illustration only, adeno-associated viral vectors will be discussed in details herein. Typically, AAV genome comprises, at its 3′ end and 5′ ends, terminal repeats which are about 145 by long. Between these repeats, the AAV contains two regions: rep, which encodes proteins controlling viral replication structural gene expression and integration into the host genome, and cap, which encodes capsid structural proteins. The total length of the AAV genome is about 4.7 kb. In different embodiments, varying parts of the AAV genome are replaced with a construct containing the bioactive nucleic acid sequence (preferably included into an expression cassette) and the inert DNA sequence of the instant invention. For example, in one embodiment, the DNA construct comprises the bioactive nucleic acid sequence and the inert DNA sequence flanked by the terminal repeats of the AAV. In another embodiment, only the cap region of the AAV genome is replaced with the DNA construct according to any embodiment described above. Accordingly, the resulting construct comprises a first part containing the nucleic acid sequence, a second part comprising the inert DNA sequence, a rep region, and two terminal repeats at the 3′ end and the 5′ end of the resulting construct, respectively. In all of these embodiments, the length of the final DNA construct is optimized for the efficient packaging and varies from about 3 kb to about 5 kb, more preferably, from about 4 kb to about 5 kb, more preferably from about 4.5 kb to about 5 kb, most preferably, about 4.7 kb. Thus, a person of ordinary skill in the art will appreciate that more than one inert DNA sequence may be used to achieve the desired length of the DNA construct. By, the same logic, in some embodiments it may be desirable to use only a part of the inert DNA sequence.

According to one embodiment the siNA expression cassette has a length of about 1 kb. The practitioner may chose to remove all genome of a viral vector (e.g., adeno associated viral vector) except the terminal repeats which have a total length of about 0.3 kb, and the desired length of the DNA construct is about 4.7 kb. This embodiment requires about 3.4 kb of inert DNA sequence. For simplicity, assuming that the inert DNA sequences in possession of the practitioner all have length of about 1 kb, the practitioner may choose to join three of these inert DNA sequences and also prepare a partial inert DNA sequence which has length of about 0.4 kb. The preparation of the partial inert DNA sequence is easily achieved by PCR with the primers selected to amplify a portion of the full length inert DNA sequence, which portion is about 0.4 kb long. Of course, multiple other ways exist for preparing this 3.4 kb long portion of the DNA construct.

Essentially, preparation of the DNA constructs according to any of the embodiments described above may be achieved by routine molecular biology techniques, including, without limitation, those, described in Ausubel et al., Current Protocols in Molecular Biology, 5th ed., Wiley Interscience (2002), incorporated herein by reference, including, without limitation, any combination of PCR, restriction endonuclease digestion, ligation, and subcloning.

In another set of embodiments, the DNA construct is packaged with a capsid, particularly, AAV capsid, thus producing recombinant AAV (rAAV) vectors. For example, the AAV expression vector which harbors the DNA construct according to any embodiment of the instant invention bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414, 5,139,941, and 7,112,321; International Publication Nos. WO 92/01070 (published 23 Jan. 1992) and WO 93/03769 (published 4 Mar. 1993); Lebkowski et al. (1988) Molec. Cell. Biol. 8:3988 3996; Vincent et al. (1990) Vaccines 90 (Cold Spring Harbor Laboratory Press); Carter (1992) Current Opinion in Biotechnology 3:533 539; Muzyczka (1992) Current Topics in Microbiol. and Immunol. 158:97 129; Kotin (1994) Human Gene Therapy 5:793 801; Shelling and Smith (1994) Gene Therapy 1:165 169; and Zhou et al. (1994) J. Exp. Med. 179:1867 1875.

One method that has been used to produce recombinant AAV (rAAV) vectors comprises co-transfecting eukaryotic cells with a plasmid containing rAAV sequences e.g., the DNA construct according to any of the embodiments described above (the cis plasmid) and a plasmid containing rep and cap (the trans plasmid), and infecting the cells with a helper virus (e.g., adenovirus or herpes virus). See U.S. Pat. No. 5,753,500. This method may be modified by altering the translation initiation codon of the Rep78/68 proteins in the trans plasmid to decrease the translation of the Rep protein and increase production of rAAV. Li et al. (J. Virol. 71:5236 5243, 1997).

The deleted rep and cap sequences are supplied to the host cells by other viruses or plasmids where they are transiently or stably expressed. There are also a number of cell lines that stably express rep and cap. The host cells also require helper functions in order for the rAAV to replicate and excise from the host cell genome. The helper functions usually are provided by helper viruses (either wild type or crippled viruses), plasmids containing the helper virus functions or physical methods.

A second method that has been used to produce rAAV involves co-transfection of three plasmids into eukaryotic cells. In this method, one plasmid carries the transgene and ITRs (the cis plasmid), a second plasmid encodes the rep and cap genes (the trans plasmid), and the third plasmid encodes the helper virus functions, i.e. adenoviral genes such as E1a, E1b, E2a and E4 (the helper plasmid).

A third method involves the use of a packaging cell line such as one including AAV functions rep and cap. See U.S. Pat. Nos. 5,658,785 and 5,837,484 and PCT US98/19463. The packaging cell line may be transfected with a cis plasmid comprising the transgene and ITRs, and infected by wild-type adenovirus (Ad) helper. See U.S. Pat. No. 5,658,785. Alternatively, the packaging cell line may be co-infected by a hybrid Ad/AAV, in which a hybrid Ad vector carries the cis plasmid in the E1 locus (see U.S. Pat. No. 5,856,152), and by a wild-type or mutant Ad that supplies E1.

For the purposes of the invention, suitable host cells for producing rAAV virions from the AAV expression vectors include microorganisms, yeast cells, insect cells, and mammalian cells, that can be, or have been, used as recipients of a heterologous DNA molecule and that are capable of growth in suspension culture. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) are preferred in the practice of the present invention. Particularly, the human cell line 293 is a human embryonic kidney cell line that has been transformed with adenovirus type-5 DNA fragments (Graham et al. (1977). J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof. AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822 3828; and McCarty et al. (1991) J. Virol. 65:2936 2945. A number of other vectors have been described which encode Rep and/or Cap expression products. See, e.g., U.S. Pat. No. 5,139,941.

The host cell (or packaging cell) must also be rendered capable of providing non-AAV-derived functions, or “accessory functions,” in order to produce rAAV virions. Accessory functions are non-AAV-derived viral and/or cellular functions upon which AAV is dependent for its replication. Thus, accessory functions include at least those non-AAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241 247; McPherson et al. (1985) Virology 147:217 222; Schlehofer et al. (1986) Virology 152:110 117.

Alternatively, accessory functions can be provided using an accessory function vector as defined above. See, e.g., U.S. Pat. No. 6,004,797 and International Publication No. WO 01/83797, incorporated herein by reference in its entirety. Nucleic acid sequences providing the accessory functions can be obtained from natural sources, such as from the genome of an adenovirus particle, or constructed using recombinant or synthetic methods known in the art. As explained above, it has been demonstrated that the full-complement of adenovirus genes are not required for accessory helper functions.

In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol 41:868; Janik et al., (1981) Proc. Natl. Acad. Sci. USA 78:1925; Carter et al., (1983) Virology 126:505. Other characterized Ad mutants include: E1B (Laughlin et al. (1982)., supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206 210, recently reported that E1B55k is required for AAV virion production, while E1B19k is not. In addition, International Publication WO 97/17458 and Matshushita et al., (1998) Gene Therapy 5:938 945, describe accessory function vectors encoding various Ad genes.

In one embodiment, accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as column chromatography, CsCl gradients, and the like. For example, a plurality of column purification steps can be used, such as purification over an anion exchange column, an affinity column and/or a cation exchange, column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

In one embodiment of the present invention, the composition comprising the DNA construct according to any of the embodiments described above (e.g., packaged into a viral capsid) is formulated in accordance with standard procedure as a pharmaceutical composition adapted for delivered administration to human beings and other mammals.

Those of skill in the art are familiar with the principles and procedures discussed in widely known and available sources as Remington's Pharmaceutical Science (17th Ed., Mack Publishing Co., Easton, Pa., 1985) and Goodman and Gilman's The Pharmaceutical Basis of Therapeutics (8th Ed., Pergamon Press, Elmsford, N.Y., 1990) both of which are incorporated herein by reference.

Typically, compositions for intravenous administration are solutions in sterile isotonic aqueous buffer. Where necessary, the composition may also include a solubilizing agent and a local anesthetic to ameliorate any pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

In cases other than intravenous administration, the composition can contain minor amounts of wetting or emulsifying agents, or pH buffering agents. The composition can be a liquid solution, suspension, emulsion, gel, polymer, or sustained release formulation. The composition can be formulated with traditional binders and carriers, as would be known in the art. Formulations can include standard carriers such as pharmaceutical grades of mannitol, lactose, starch, magnesium stearate, sodium saccharide, cellulose, magnesium carbonate, etc., inert'carriers having well established functionality in the manufacture of pharmaceuticals. Various delivery systems are known and can be used to administer a therapeutic of the present invention including encapsulation in liposomes, microparticles, microcapsules and the like.

In yet another preferred embodiment, the DNA construct according to any embodiment of the invention can be formulated as neutral or salt forms. Pharmaceutically acceptable salts include those formed with free amino groups such as those derived from hydrochloric, phosphoric, acetic, oxalic, tartaric acids and the like, and those formed with free carboxyl groups such as those derived from sodium, potassium, ammonium, calcium, ferric hydroxides, isopropylamine, thriethylamine, 2-ethylamino ethanol, histidine, procaine or similar.

The amount of the DNA construct which will be effective in the treatment of a particular disorder or condition will depend on the nature of the disorder or condition, and can be determined by standard clinical techniques, well established in the administration of therapeutics. The precise dose to be employed in the formulation will also depend on the route of administration, and the seriousness of the disease or disorder; and should be decided according to the judgment of the practitioner and the patient's needs. Suitable dose ranges for intracranial administration are generally about 10³ to 10¹⁵ infectious units of the DNA construct per microliter delivered in 1 to 3000 microliters of single injection volume. Addition amounts of infections units of vector per micro liter would generally contain about 10⁴, 10⁵, 10⁶, 10⁷, 10⁸, 10⁹, 10¹⁰, 10¹¹, 10¹², 10¹³, 10¹⁴ infectious units of viral vector delivered in about 10, 50, 100, 200, 500, 1000, or 2000 microliters. Effective doses may be extrapolated from dose-responsive curves derived from in vitro or in vivo test systems.

For the application of the DNA construct according to any of the embodiments described above for treatment of different neurodegenerative diseases, multiple catheters having access ports can be implanted in a given patient for a complete therapy. In a preferred embodiment, there is one port and catheter system per cerebral or cerebellar hemisphere, and perhaps several. Once the implantations are performed by a neurosurgeon, the patient's neurologist can perform a course of therapy consisting of repeated bolus injections of the DNA construct of the instant invention over a period of weeks to months, along with monitoring for therapeutic effect over time, The devices can remain implanted for several months or years for a full course of therapy. After confirmation of therapeutic efficacy, the access ports might optionally be explanted, and the catheters can be sealed and abandoned, or explanted as well. The device material should not interfere with magnetic resonance imaging, and, of course, the formulations comprising the DNA constructs must be compatible with the access port and catheter materials and any surface coatings.

In one embodiment, the route of delivery is through the use of implanted, indwelling, intraparenchymal catheters that provide a means for injecting small volumes of fluid containing the DNA construct according to any embodiment of the instant invention directly into local brain tissue. The proximal end of these catheters may be connected to an implanted, intracerebral access port surgically affixed to the patient's cranium, or to an implanted drug pump located in the patient's torso.

Examples of the delivery devices within the scope of the present invention include the Model 8506 investigational device (by Medtronic, Inc. of Minneapolis, Minn.), which can be implanted subcutaneously on the cranium, and provides an access port through which therapeutic agents may be delivered to the brain. Delivery occurs through a stereotactically implanted polyurethane catheter. Two models of catheters that can function with the Model 8506 access port include the Model 8770 ventricular catheter by Medtronic, Inc., for delivery to the intracerebral ventricles, which is disclosed in U.S. Pat. No. 6,093,180, incorporated herein by reference, and the IPA1 catheter by Medtronic, Inc., for delivery to the brain tissue itself (i.e., intraparenchymal delivery), disclosed in U.S. Ser. Nos. 09/540,444 and 09/625,751, which are incorporated herein by reference. The latter catheter has multiple outlets on its distal end to deliver the therapeutic agent to multiple sites along the catheter path. In addition to the aforementioned device, the delivery of the DNA constructs in accordance with the present invention can be accomplished with a wide variety of devices, including but not limited to U.S. Pat. Nos. 5,735,814, 5,814,014, and 6,042,579, all of which are incorporated herein by reference. Using the teachings of the present invention and those of skill in the art will recognize that these and other devices and systems may be suitable for delivery of small interfering RNA vectors for the treatment of neurodegenerative diseases in accordance with the present invention.

In one preferred embodiment, the method further comprises the steps of implanting a pump outside the brain, the pump coupled to a proximal end of the catheter, and operating the pump to deliver the predetermined dosage of the DNA construct through the discharge portion of the catheter. A further embodiment comprises the further step of periodically refreshing a supply of the DNA construct to the pump outside the brain.

The pre-determined location of the brain may be mapped by many methods. For example, for some application, the targeted area may be located by stereotactical or gross anatomical atlases. In other embodiments, when the precise location of the targeted area is crucial, e.g., when the deliverable amount of the DNA construct is delivered into the brain of the patient, other mapping means may be used. Such mapping means include, without limitation, Positron Emission Tomography and Single Photon Emission Computed Tomography (PET and SPECT, respectively), pharmacological Magnetic Resonance Imaging (phMRI), functional MRI (fMRI), and contrast-enhanced computerized tomography (CT) scan.

In another embodiment, Computer-aided atlas-based functional neurosurgery methodology can be used to accurately and precisely inject the deliverable amount of the DNA construct of the present invention. Such methodologies permit three-dimensional display and real-time manipulation of cerebral structures. Neurosurgical planning with mutually preregistered multiple brain atlases in all three orthogonal orientations is therefore possible and permits increased accuracy of target definition for therapy injection or implantation, reduced time of the surgical procedure by decreasing the number of tracts, and facilitates planning of more sophisticated trajectories. See e.g. Nowinski W. L. et al., Computer-Aided Stereotactic Functional Neurosurgery Enhanced by the Use of the Multiple Brain Atlas Database, IEEE Trans Med Imaging 19(1); 62-69:2000.

Preferably, the pre-determined target area in the brain of the patient is determined on an individual basis, e.g., by real time image guidance, so that the neurosurgeon will see exactly where the catheter is being placed. Suitable systems exist for this particular embodiment, including, without limitation, STEALTH station developed by Surgical Navigation Technologies, a division of Medtronic. This tool incorporates preoperative images, including MRI, CT, and functional imaging studies into the computers in the operating room. A hand held probe linked to the computer can be used to point anywhere on the patients head or brain, with the corresponding area shown with great accuracy on a computer screen. Thus, there is no need to guess at the relationship between an area on or in the brain, inspected by sight and where that corresponds to the patient's preoperative images.

In additional aspects, the invention provides a medical system comprising an intracranial access device according to any of the embodiments described above (e.g., the intracranial access port), a mapping means according to any of the embodiments described above (including, without limitation, image-guided mapping means); at least one of the DNA construct or the composition according to any embodiment described above; and a delivery means according to any embodiment described above. Thus, this aspect of the invention provides a solution to at least two problems present in the art: first, the DNA construct or the composition which is suitable for efficient packaging into viral vectors and safe for the patient, and second, the system of the instant invention solves a problem of delivery of the siRNA to the target areas.

In yet another aspect, the invention provides a kit comprising the DNA construct and/or the composition according to any of the embodiments described above, and a set of instructions comprising information for safe and efficient use of the DNA construct and/or the composition. In a selected embodiment, the kit may further comprise at least one of an intracranial access port; mapping means for locating a pre-determined target area in a brain of a patient, said predetermined target area comprising cells natively expressing a gene involved in a neurodegenerative disorder; and a delivery means. A person of ordinary skill in the art will undoubtedly appreciate that the mapping means (e.g., a Stealth station or other mapping means) can be re-used with many patients. On the other hand, the delivery means (e.g., catheter or catheter-pump combination) and the intracranial access device are single-patient devices. Thus in a specific embodiment, the kit comprises, the intracranial access device, the delivery means, the set of instructions and the DNA construct and/or the composition according to any of the embodiments described above.

The invention will now be further described in the following non-limiting examples.

### EXAMPLES

### Example 1

### Construction of pAAV-shRNA-InertDNA Plasmids

1) Modification of Polylinker in pBlueScript KS+(Stratagene)

a) Cut pBlueScript KS+ with BamHI and KpnI

b) oligos STF-A and STF-B were annealed and ligated into the digested vector. The oligos were designed to have sticky ends complementary to the cut vector.

STF A:

(SEQ ID NO: 16)

gatcacgcgtaggcctagaattcattctcgagtatggtacctcaggatcc
cggaccgagtac
STF-B:

(SEQ ID NO: 17)

tcggtccgggatcctgaggtaccatactcgagaatgaattctaggcctac
gcgt

This resulted in a new vector with a modified poly linker containing restriction sites in the following order MluI-StuI-EcoRI-XhoI-KpnI-BamHI-RsrII

2) Using the PCR primers tailed with Restriction endonuclease sites EcoRI and XhoI a 1092 bp fragment was amplified from disclosed sequence 2_I 1285 (SEQ. ID. NO. 4). This was digested with EcoRI and XhoI and ligated into modified pBlueScript cut with the same enzymes.

| | |
|---|---|
| Stuffer A FOR: | |
| GAATTCTCTTTTGATGTATAATATTTTAA | (SEQ ID NO: 18) |
| Stuffer A REV: | |
| CTCGAGAGTGAAGAATAAAGGAAAA | (SEQ ID NO: 19) |

3) Using the PCR primers tailed with Restriction endonuclease sites KpnI and BamHI a 1257 bp fragment was amplified from disclosed sequence 2_F 1245 (SEQ ID NO: 12). The additional 12 bp result from the addition of the two restriction sites to the 1245 bp fragment. This was digested with KpnI and BamHI and ligated into modified pBlueScript cut with the same enzymes.

| | |
|---|---|
| Stuffer C for: | |
| GGTACCTTTACTCAGGAGCTTTTG | (SEQ ID NO: 20) |
| Stuffer C REV: | |
| GGATCCTTTGTAGAAATAAAGCAATA | (SEQ ID NO: 21) |

4) Using a PCR primer tailed with an XhoI restriction site (B for 3: CTCGAGTGACAGGTTGCCAGAGA, SEQ ID NO: 22) and a second primer (B rev 3a: GCAGAAGTTCAGGCCACAGTTG, SEQ ID NO:23) a 749 bp fragment was PCR amplified from the disclosed sequence 3_H 1990 (SEQ ID NO: 7). This PCR fragment was digested with XhoI and NcoI to generate a 674 bp fragment. The NcoI site lies within the amplified region.

5) Using the PCR primers tailed with Restriction endonuclease sites NcoI and KpnI a 791 bp fragment was amplified from disclosed sequence 3_I 1408 (SEQ ID NO: 9). This was digested with NcoI and KpnI.

| | |
|---|---|
| For D: | |
| CCATGGAATAGTGATTCACAATTTTATCAT | (SEQ ID NO: 24) |
| Rev D: | |
| GGTACCTTAAGGACCTAGACTATCCAAAGC | (SEQ ID NO: 25) |

6) The vector from step 3 was cut with XhoI and KpnI. A triple ligation was performed using the XhoI-NcoI fragment from step 4 and the NcoI-KpnI fragment from step 5. The final inert. DNA sequence of 3796 bp (INERTverC, SEQ ID NO: 15) is contained between the EcoRI and BamHI sites of this vector.

The analysis of the INERTverC sequence, as described above, revealed that this sequence did not have a pol II promoter, did not have CpG islands, did not have predicted splice donor and/or acceptor sites with high probability scores, and did not have a standard ORF. The fragments used for the INERTverC sequence (2_I 1285; 3_H 1990, 3_I 1408, and 2_F 1245) revealed 94%, 91%, 92%, and 86% homology with the rhesus genomic database.

7) The MluI-RsrII fragment from this plasmid (containing the inert DNA) was subcloned into pAAV-hrGFP (Stratagene) and the corresponding sites.

8) Expression cassettes containing the human U6 promoter and shRNA sequences were removed from previously disclosed plasmids (US2006/0257912 A1) using restriction endonucleases BglII and EaeI: The fragment was treated with T4 DNA polymerase to generate a blunt-ended fragment of 376 bp.

9) This blunted-ended expression cassette described in step 8 was subcloned into StuI cut vector described in step 7.

10) The identity of the HD-1, HD-5, CTRL-1, and CTRL-5 plasmids was confirmed by restriction endonuclease digestion and sequence analysis.

### Example 2

### Addition of Inert DNA Sequences of the Instant Invention does not Affect the Extent and the Specificity of Attenuation of Huntingtin mRNA by shRNA

Four shNA constructs were used in this study. These constructs are designated as follows:

HD-1 (passenger strand - TGACAGCAGTGTTGATAAA,
SEQ ID NO: 26):

expresses an shRNA against the human/rhesus Huntington gene (targets both rhesus and human HD)

CTRL-1
(passenger strand-TGACGAAGTCGTGATTAAA, SEQ ID NO:
27):

expresses a scrambled version of HD-1

HD-5 (passenger strand - GGAGTATTGTGGAACTTAT,
SEQ ID NO: 28):

expresses an shRNA against the human/rhesus Huntington gene
(targets both rhesus and human HD)

CTRL-5
(passenger strand-GGAGTAGTCGTAATGTTAT, SEQ ID NO:
29):

expresses a scrambled version of HD-5.

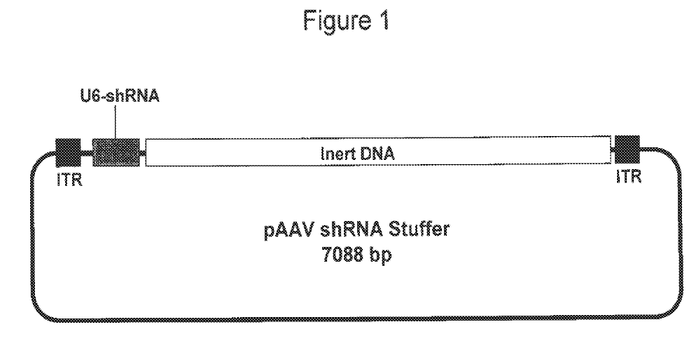

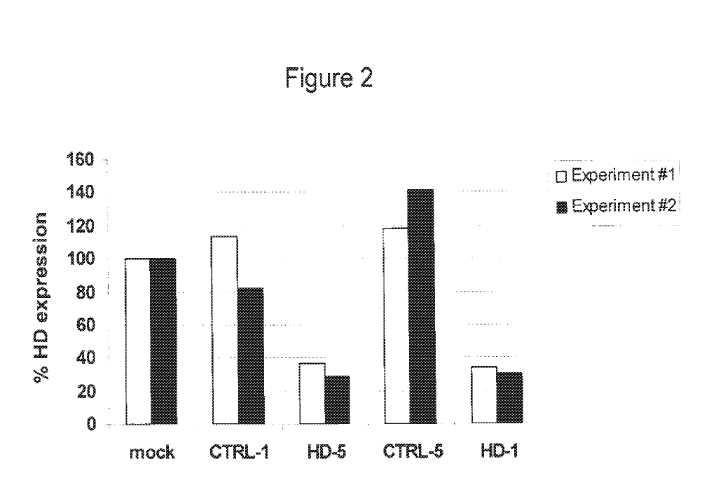

These constructs were incorporated into the construct illustrated in FIG. 1, and the inert DNA sequence was identical to SEQ. ID. NO. 15. The plasmids were transfected into HEK293T cells using Transit-293 transfection reagent (Mirus Bio, Madison, Wis.). RNA was collected 48 hours post transfection and was used to generate cDNA by standard methods. The suppression of endogenous human HD gene expression was measured by real-time PCR and normalized to the amount of endogenous human GAPDH expressed. Normalized human huntingtin mRNA expression was then compared to the level observed in mock transfected cells. As can be seen in FIG. 2, pAAV plasmids containing U6-HD-1 and U6-HD-5 were effective at suppressing endogenous human huntingtin mRNA expression. pAAV plasmids expressing scrambled control sequences did not suppress endogenous human huntingtin mRNA expression. The data illustrated in FIG. 2 reflect the average of two independent experiments, each is the average of data collected by two investigators.

### Example 3

### In Vitro Validation of rAAV Expressing shRNA Against HD and Containing Inert DNA Sequence (INERTverC)

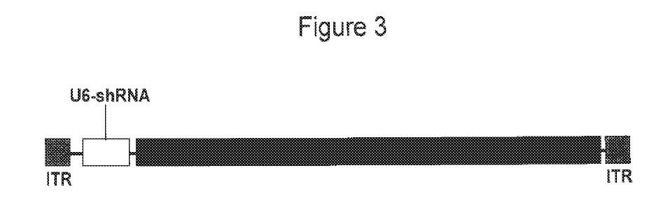

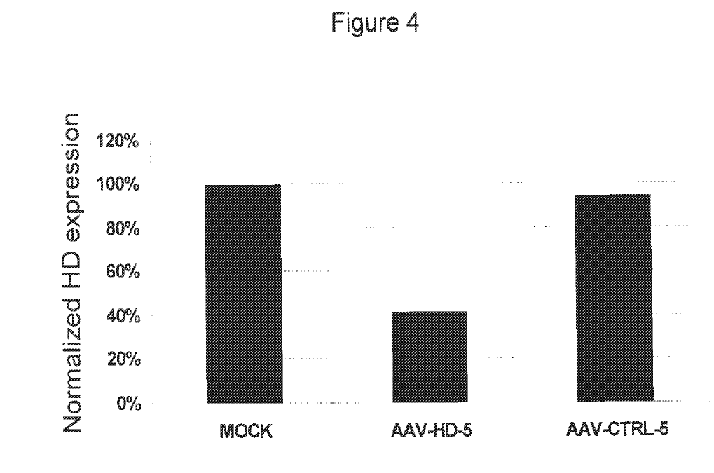

The described pAAV plasmids along with the required pHELPER and REPCAP plasmids were used to generate recombinant adeno-associated virus (AAV) with serotype 1. The schematic illustration of AAV genome is shown in FIG. 3. Two separate AAV viruses were generated. These viruses were engineered to express either an shRNA targeting Huntington (AAV-HD-5) or a scrambled version of this shRNA that does not target Huntington (AAV-CTRL-5). This is a scrambled version of AAV-HD-5. Expression of both of these shRNAs is driven by the human U6 promoter. The ability of these viruses to suppress endogenous human HD gene expression in HEK293T cells was examined. 5*10⁵ cells in a well of a 6-well plate were transduced with 5*10⁹ virions by directly adding the virus to the well of cells in 1 mL of Dulbecco's Modified Eagle Medium (DMEM) cell culture media supplemented with 2% fetal bovine serum (FBS). This plate was incubated at 37° C., 5% CO₂ for 2 hours with gentle mixing by rocking every 30 minutes. Following this 2-hour incubation, 1 mL of DMEM cell culture media supplemented with 18% FBS was added to each well yielding a final FBS concentration of 10%. Growth of the transduced cells was continued at 37° C., 5% CO₂. RNA was collected 72 hours post transduction and used to make cDNA by standard methods. The cDNAs were analyzed for HD and GAPDH expression levels by real-time PCR methods. Endogenous HD expression was normalized to endogenous GAPDH expression. As shown in FIG. 4, compared to mock transduced cells, AAV-HD-5 was effective in suppressing endogenous Huntington while AAV-CTRL-5 was unable to suppress Huntington expression.

### Example 4

### INERT DNA Sequence does not Interfere with AAV Packaging

A standard method to quantify the number of viral particles in an AAV preparation is to analyze viral capsid proteins (VP1, VP2 and VP3) using polyacrylamide gel electrophoresis. The gel is then silver stained and the amount of each capsid protein is determined using densitometry by comparing to known standard amounts of viral capsid proteins. Using this method for an adeno-associated virus expressing the red fluorescent protein (RFP) and also containing a portion of the INERTverC sequence (AAV1-RFP:INERT), as described in example 9, the number of viral particles per milliliter was found to be 2*10¹². This method is unable to distinguish between empty viral capsids and those containing a complete viral genome.

To determine if the INERT DNA sequence affected the efficiency of viral packaging the viral titer of AAV1-RFP:INERT was determined using a PCR-based absolute quantification method. To accomplish this, the number of copies of viral genomic DNA in a sample is quantified using a PCR standard curve made up of samples containing known copies of DNA. The number of viral genomes per milliliter for AAV1-RFP:INERT was determined to be 4.8±0.09*10¹². Slight differences in the values determined by these two methods can be attributed to the sensitivity of each assay method. From this experiment it can be concluded that INERT DNA sequences do not affect the efficiency of AAV packaging.

### Example 5

### In Vitro Validation of rAAV Serotype 1 Expressing shRNA Against Hd and Containing Inert DNA Sequence (INERTverC)

Similar to example 3, pAAV plasmids along with the required pHELPER and REPCAP plasmids were used to generate recombinant adeno-associated virus (AAV) with serotype 1. Two separate AAV viruses were generated. These viruses were engineered to express either a shRNA targeting Huntington (AAV1-HD-1) or a scrambled version of this shRNA that does not target Huntington (AAV1-CTRL-1). This is a scrambled version of AAV1-HD-1. Expression of both of these shRNAs is driven by the human U6 promoter. The ability of these viruses (as well as AAV1-HD-5 and AAV1-CTRL5) to suppress endogenous human HD gene expression in HEK293T cells was examined. The methods used to quantify endogenous HD expression are as those described in example 3. The level of endogenous HD expression was normalized to that obtained in cells transduced with a virus expressing the green fluorescent protein (AAV1-GFP). In multiple experiments, both AAV1-HD-5 and AAV1-HD-1 were effective in suppressing endogenous Huntington while AAV1-CTRL-5 and AAV1-CTRL-1 were unable to suppress Huntington expression.

### Example 6

### In Vitro Validation of rAAV Serotype 2 Expressing shRNA Against Hd and Containing Inert DNA Sequence (INERTverC)

The described pAAV plasmids along with the required pHELPER and REPCAP plasmids were used to generate recombinant adeno-associated virus (AAV) with serotype 2. Two separate AAV viruses were generated. A third virus generated using the pAAV-hrGFP plasmid (Stratagene, La Jolla, Calif.) was used as a control (AAV2-hrGFP). The ability of AAV2-HD-5 and AAV2-CTRL-5 to suppress endogenous human HD gene expression in HEK293T cells was examined. The methods used are as those described in example 3. As shown, compared to AAV2-hrGFP transduced cells, AAV2-HD-5 was effective in suppressing endogenous Huntington while AAV2-CTRL-5 was unable to suppress Huntington expression. These results indicate that rAAV of either serotype 1 or serotype 2 containing INERT DNA sequence (INERTverC) can be efficiently packaged. Moreover, both of these serotypes are able to express a functional shRNA able to suppress its target gene.

### Example 7

### Huntington mRNA Suppression in Rhesus Monkey (AAV2-HD5)

In this study, a 15 year old female rhesus monkey weighing approximately 8.0 kg was anesthetized and bilaterally co-infused with AAV2-HD-5 and AAV2-hrGFP by stereotactic injection. Each hemisphere received 1.5*10¹¹ viral particles of each rAAV (75 μl of each virus, 150 μl total). Stereotactic targets were determined by presurgical MRI and included rostral putamen (AP=21 mm; 14.11.5 mm; DV1=23.5 mm and DV2=20.5 mm) caudal putamen (AP=17 mm; L=14 mm; DV1=23.5 mm and DV2=20.5 mm) and caudate nucleus (AP=24 mm; L=6 mm; DV=18 mm) sites. Injections (Hamilton syringe (150 μl) with 27 Ga needle (compression fitting)) were performed at 4 putamen sites (30 μl/site) using two needle tracts and 1 caudate site (30 μl) at a rate of 2 μl/min. Following this injection, the needle was left in place for at least 20 minutes and then withdrawn at a rate of 1 mm/min.

Twenty-eight days later, the animal was deeply anesthetized with pentobarbital (1.5 ml intramuscular; 2 ml, intravenous) and transcardially perfused with heparinized ice-cold saline (4-6 L). Following perfusion and removal, the brain was placed into a container of ice-cold saline.

One hemisphere of the brain was sectioned into blocks. Each block was embedded in OCT, snap frozen in isopentane and stored at −80° C. Ten micron sections were then collected and placed onto laser microdissection (LMD) compatible slides. Tissue on the slides was fixed in ethanol (30 seconds in 75% ethanol; 30 seconds in 95% ethanol; and 30 seconds in 100% ethanol) with an air dry between steps. Slides were then screened to identify the ones containing brain regions of interest (i.e. caudate and putamen) and data on viral spread was obtained by following the expression of the marker gene (GFP). GFP positive regions were collected by LMD from the caudate and putamen.

RNA was isolated from GFP collected material using the PicoPure RNA isolation kit from Arcturus (Mountain View, Calif.). The RNA was quantified prior to the generation of cDNA. Typically about 100-300 ng of total RNA from the LMD collected material was isolated. cDNA was prepared using the AffinityScript® QPCR cDNA synthesis kit from Stratagene (La Jolla, Calif.). To generate cDNA 20 ng of total RNA was used in each cDNA synthesis reaction. HD expression was measured by realtime PCR and normalized to GAPDH or 18S rRNA expression in the same sample. To verify RNA collected from virally transduced cells were being analyzed, GFP realtime PCR to monitor GFP expression was also performed. As shown, in two independent LMD collections AAV2-HD-5 was effective in suppressing endogenous Huntington. The relative level of suppression was determined by normalizing HD expression to either GAPDH or 18S rRNA and comparing the HD expression to that measured in the LMD sample collected from a similar brain region in which expression of AAV-hrGFP was not detected.

### Example 8

### Effects of Long-Term Expression of rAAV Serotype 2 Containing Inert DNA (INERTverC) in Mice

In this study, mice were bilaterally infused with AAV2-HD-5 (n=5), AAV2-CTRL-5 (n=5), PBS (n=5), AAV2-hrGFP (n=4) or AAV1-GFP (n=4) by stereotactic injection. Each hemisphere received 1.0*10¹⁰ viral particles of each rAAV (5 μl virus) or 5 μl of PBS. The striatal stereotactic target was determined using a mouse brain atlas (AP=0.5 mm; ML=1.75 mm; DV=3.6 mm). Bilateral injections were performed using a Hamilton syringe with 33 Ga needle at a rate of 0.5 μl/min. Of note, the viruses expressing shRNAs (AAV2-HD-5 and AAV2-CTRL-5) are expected to be innocuous in mice due to non-homologies in the murine HD sequence. These shNAs were designed to target both human and rhesus monkey Huntington. In this case, there is at least 1 non-homology in the murine Huntington sequence within each of the HD-1 and HD-5 target sequences. Sixteen weeks post surgery rotarod analyses were performed. This is a sensitive test of motor coordination. In this test the time a mouse is able to stay on an accelerating rod is determined. The mice are tested on three successive days and given four trials on each of those days. As shown by the rotarod analysis, the performance of mice expressing either AAV2-HD-5 or AAV2-CTRL-5 for sixteen weeks is similar to that of mice expressing AAV1-GFP, AAV2-hrGFP or PBS. Repeated measures ANOVA analysis found no statistical difference among the groups tested. This indicates long-term expression of rAAV containing INERT DNA (INERTverC) does not result in detectable motor deficits as measured by rotarod analysis.

### Example 9

### Inclusion of INERT DNA on a Plasmid or Within a rAAV Viral Genome does not Interfere with the Expression of the Red Fluorescent Protein (RFP) Reporter Gene

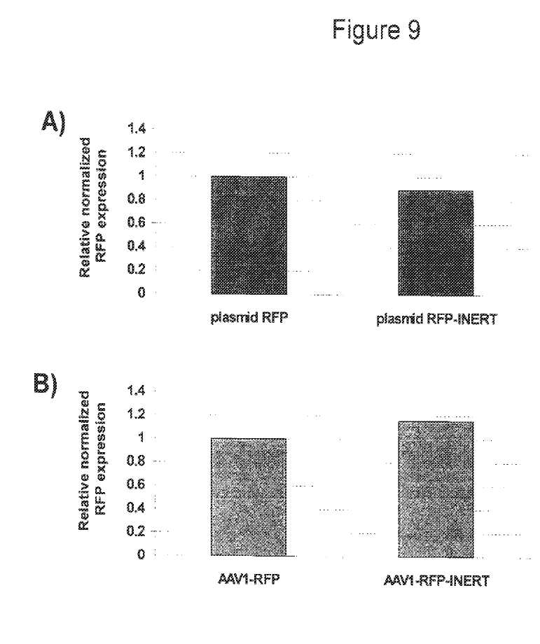

A pAAV plasmid expressing the red fluorescent protein (RFP) was constructed with and without INERT DNA. The pAAV-RFP plasmid had a second expression cassette unrelated to RFP, resulting in a predicted viral genome size near that of wild-type AAV. The addition of the INERT DNA to pAAV-RFP:INERT brought the predicted size of the viral genome near that of wild-type AAV (4.7 kB). The INERT DNA used to construct this pAAV plasmid contained approximately 2.4 kB of INERTverC (NcoI to BamHI). To determine if the inclusion of the INERT DNA affected the level of RFP transcription these plasmids were transfected into HEK293T cells and the level of RFP expression was determined by quantitative realtime PCR. To do this, the plasmids were transfected into HEK293T cells using Transit-293 transfection reagent (Mirus Bio, Madison, Wis.). For each transfection 2 μg of the relevant plasmid was used. RNA was collected 48 hours post transfection and was used to generate cDNA by standard methods. The level of RFP expression was measured by real-time PCR and normalized to the amount of endogenous human GAPDH expressed. Normalized RFP mRNA expression in cells transfected with pAAV-RFP was then compared to the level observed in cells transfected with pAAV-RFP:INERT. As can be seen in FIG. 9A, the normalized level of RFP expression is similar for both plasmids. This indicates the INERT DNA does not interfere with the expression of the RFP reporter gene.

To further this analysis the described pAAV plasmids along with the required pHELPER and REPCAP plasmids were used to generate recombinant adeno-associated virus (AAV) with serotype 1. Two separate AAV viruses were generated, AAV1-RFP and AAV1-RFP:INERT. The level of RFP expression from these viruses was assessed in vitro in HEK293T cells. The methods used for this analysis are described in example 3. Of note, the same number of viral particles was used for both AAV1-RFP and AAV1-RFP:INERT. The cDNAs were analyzed for both RFP and GAPDH expression levels by realtime PCR methods. Expression of RFP from the virus was normalized to endogenous GAPDH expression. As shown in FIG. 9B, the level of virally expressed RFP is similar for AAV1-RFP and AAV1-RFP:INERT. This indicates in the context of rAAV INERT DNA does not interfere with the expression of the RFP reporter gene.

Although the invention herein has been described with reference to particular embodiments, it is to be understood that these embodiments are merely illustrative of the principles and applications of the present invention. For example, a person of ordinary skill in the art will appreciate that the concepts of the DNA constructs according to any embodiment of the instant invention, as well as methods and systems for delivery of these DNA constructs can be applied to other diseases, including, without limitations, diseases of the myocardium, peripheral nervous system, organs (diabetes), diseases of the spine and joints, and complex diseases such as obesity without excessive experimentation. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

1. An isolated inert DNA sequence having a length of between about 0.5 kb and about 5 kb, wherein said isolated inert DNA sequence does not contain an open reading frame.

2. The isolated inert DNA sequence of claim 1, wherein said isolated inert DNA sequence does not contain a polII promoter.

3. The isolated inert DNA sequence of claim 1, wherein said isolated inert DNA sequence contains no CpG islands.

4. The isolated inert DNA sequence according to claim 1, wherein said isolated inert DNA sequence does not contain a splice donor site or a splice acceptor site.

5. The isolated inert DNA sequence according to claim 1, wherein said isolated inert DNA sequence does not contain a miRNA sequence.

6. The isolated inert DNA sequence of claim 1, wherein said isolated inert DNA sequence does not include a histone binding site.

7. The isolated inert DNA sequence of claim 1, wherein said isolated inert DNA sequence does not include an imprinting center.

8. The isolated inert DNA sequence according to claim 1, having at least a portion which is at least 75% identical to a portion of a genome of a mammal of family Hominidae or family Cercopithecidae.

9. The isolated inert DNA sequence according to claim 8, wherein the mammal is a human.

10. The isolated inert DNA sequence according to claim 9, wherein at least the portion is 100% identical to the portion of the genome of the human.

11. An isolated inert DNA sequence comprising a sequence selected from the group consisting of SEQ. ID. NO. 1 to SEQ. ID. NO. 15, and any combination thereof.

12. A DNA construct comprising:
a) a first part comprising a nucleic acid sequence encoding a bioactive nucleic acid; and
b) a second part comprising at least a portion of the isolated inert DNA sequence according to claim 1 and having length of between about 0.5 kb and about 4.5 kb.

13. The DNA construct of claim 12, further comprising a third part, wherein the third part comprises at least a portion of a genome of a virus.

14. The DNA construct of claim 13, wherein the virus is adeno-associated virus and wherein the DNA construct has length of between about 3.5 kb and about 5 kb.

15. The DNA construct of claim 12, wherein first part comprises an expression cassette.

16. The DNA construct of claim 12, wherein said bioactive nucleic acid comprises an RNAi agent.

17. The DNA construct of claim 12, wherein said RNAi agent comprises SEQ. ID. NO. 28.

18. A composition comprising the DNA construct of claim 12, packaged into a viral capsid.

19. The composition of claim 18, further comprising a suitable carrier or diluent.

20. The composition of claim 18 in a sustained-release formulation.

21. The method of a selective inhibition of a target gene in a live mammal, the method comprising administering to said mammal a DNA construct comprising:
at least a portion of an isolated inert DNA sequence of claim 1, having length between about 0.5 kb and about 4.5 kb and
a nucleic acid sequence encoding a bioactive nucleic acid and optionally packaged into a viral capsid, wherein the bioactive nucleic acid cleaves the target mRNA via RNA interference.

22. A method of selecting inert DNA sequences in a genome comprising identifying target sequences having length above 0.5 kb and containing no open reading frame.

23. The method of claim 22, further comprising selecting the target sequences containing no polII promoter.

24. The method of claim 22, further comprising selecting the target sequences containing no CpG islands.

25. The method of claim 22, further comprising selecting the target sequences do not contain a splice donor site or a splice acceptor site.

26. The method of claim 22, further comprising selecting the target sequences which do not contain miRNA or miRNA precursor sequences.

27. The method of claim 22, further comprising selecting the target sequences having the greatest intraspecies variation.

28. The method of claim 22, further comprising selecting the target sequences not comprising functional histone binding sites.

29. The method of claim 22, wherein the target sequence does not comprise a functional imprinting center.

30. The method of claim 22, wherein the genome is a human genome.

31. A medical system comprising:
a) an intracranial access port;
b) mapping means for locating a pre-determined target area in a brain of a patient, said predetermined target area comprising cells natively expressing a gene involved in a neurodegenerative disorder;
c) at least one of the DNA construct according to claim 12 or the composition according to claim 17;
d) a delivery means.

32. A kit comprising:
a) at least one of the DNA construct according to claim 12 or the composition according to claim 17; and
b) a set of instructions.

33. The kit of claim 32, further comprising at least one of
c) an intracranial access port;
d) mapping means for locating a pre-determined target area in a brain of a patient, said predetermined target area comprising cells natively expressing a gene involved in a neurodegenerative disorder; and
e) a delivery means.

34. A cell comprising at least one of the DNA construct according to claim 12 or the composition according to claim 18.

35. The cell of claim 34, which is a mammalian cell.

36. The cell of claim 35, which is a human cell.

37. The cell of claim 34 which is located ex vivo.

38. A non-human mammal comprising at least one of the DNA construct according to claim 12 or the composition according to claim 18.

39. An isolated inert DNA sequence having a length of between about 0.5 kb and about 5 kb, wherein:
(a) said isolated inert DNA sequence does not contain an open reading frame;
(b) said isolated inert DNA sequence does not contain a polII promoter;
(c) said isolated inert DNA sequence contains no CpG islands;
(d) said isolated inert DNA sequence does not contain a splice donor site or a splice acceptor site;
(e) said isolated inert DNA sequence does not contain a miRNA sequence;
(f) said isolated inert DNA sequence does not include a histone binding site; and
(g) said isolated inert DNA sequence does not include an imprinting center.
